(19) United States
(12) Patent Application Publication
(10) Pub. No.: US 2022/0400960 A1
(43) Pub. Date: Dec. 22, 2022

(54) AUTOREGULATION MONITORING USING DEEP LEARNING

(71) Applicant: Covidien LP, Mansfield, MA (US)

(72) Inventors: Dean Montgomery, Edinburgh (GB); Paul S. Addison, Edinburgh (GB); Andre Antunes, Edinburgh (GB)

(21) Appl. No.: 17/354,811

(22) Filed: Jun. 22, 2021

(51) Int. Cl. A61B 5/0205; A61B 5/021; A61B 5/145; A61B 5/20; A61B 5/00

(52) U.S. Cl. CPC A61B 5/0205; A61B 5/021; A61B 5/14542; A61B 5/208; A61B 5/7267; A61B 5/742; A61B 5/201; A61B 5/7275

(57) ABSTRACT

In some examples, a system is configured to determine a non-cerebral autoregulation status value of a patient using machine learning. In some examples, processing circuitry of the system is configured to determine, using a neural network algorithm that has been trained via machine learning training, an individualized adjustment value that is individualized for the patient, including inputting physiological data associated with the patient. The processing circuitry may determine a non-cerebral autoregulation status of the patient based on a cerebral autoregulation value of the patient based on the non-cerebral autoregulation status value of the patient and the adjustment value.

### TECHNICAL FIELD

This disclosure relates to monitoring autoregulation status of a patient.

### BACKGROUND

Clinicians may monitor one or more physiological parameters of a patient, e.g., to monitor a patient's autoregulation status. Autoregulation is the response mechanism by which an organism regulates blood flow over a wide range of systemic blood pressure changes through complex myogenic, neurogenic, and metabolic mechanisms. During autoregulation, arterioles dilate or constrict in an attempt to maintain appropriate blood flow. Autoregulation may occur for a variety of organs and organ systems, such as, for example, the brain, the kidneys, the gastrointestinal tract, and the like. In the example of cerebral autoregulation, as blood pressure decreases, cerebral arterioles dilate in an attempt to maintain blood flow. As blood pressure increases, cerebral arterioles constrict to reduce the blood flow that could cause injury to the brain.

### SUMMARY

This disclosure describes example devices, systems, and techniques for determining a non-cerebral autoregulation status for a non-cerebral organ (e.g., a kidney) of a patient using machine learning. The determination of the non-cerebral autoregulation status can include, for example, determination of a value, such as a lower limit of autoregulation (LLA) or an upper limit of autoregulation (ULA) for the non-cerebral organ. In some examples, the system may determine, using a neural network algorithm that has been trained via machine learning training, an individualized adjustment value for the patient with which processing circuitry can determine a non-cerebral autoregulation status value of the patient based on a cerebral autoregulation value of the patient. The system may also determine a cerebral autoregulation status value for the patient. The system may therefore determine a non-cerebral autoregulation status value for the patient as a function of the cerebral autoregulation status value for the patient and the adjustment value that is individualized for the patient based on the patient's physiological features.

The system may provide a signal indicative of the non-cerebral autoregulation status value and/or a signal indicative of the cerebral autoregulation status values to an output device for display to a clinician. In this manner, the system may allow for concurrent display of the non-cerebral autoregulation status and the cerebral autoregulation status of a patient, without requiring invasive patient monitoring to determine the non-cerebral autoregulation status.

By using a non-cerebral autoregulation model that includes a neural network algorithm that has been trained via machine learning training to determine an adjustment value that is individualized for the patient based on at least the patient's physiological data that is used to determine the non-cerebral autoregulation status of a patient, the devices, systems, and techniques of this disclosure may enable non-cerebral autoregulation monitoring devices to more accurately determine, using an individualized adjustment value determined from the physiological data collected from the patient, the non-cerebral autoregulation status of patients with fewer false positives and false negatives compared with using rote algorithms. As such, the techniques disclosed in this disclosure provides a technical advantage.

In one example, this disclosure describes a method comprising receiving, by processing circuitry, a cerebral autoregulation status value for a patient; determining, by the processing circuitry and using a neural network algorithm of a non-cerebral autoregulation model, an adjustment value that is individualized for the patient based at least in part on physiological data associated with the patient; determining, by the processing circuitry, a non-cerebral autoregulation status value of the patient based on the cerebral autoregulation status value and the adjustment value; and sending, by the processing circuitry and to an output device, a signal indicative of the non-cerebral autoregulation status value of the patient.

In another example, this disclosure describes a system comprising: memory; and processing circuitry operably coupled to the memory and configured to: receive a cerebral autoregulation status value for a patient; determine, using a neural network algorithm of a non-cerebral autoregulation model, an adjustment value that is individualized for the patient based at least in part on physiological data associated with the patient; determine a non-cerebral autoregulation status value of the patient based on the cerebral autoregulation status value and the adjustment value; and send, to an output device, a signal indicative of the non-cerebral autoregulation status value of the patient.

In another example, this disclosure describes a non-transitory computer readable storable medium comprising instructions that, when executed, cause processing circuitry to: receive a cerebral autoregulation status value for a patient; determine, using a neural network algorithm of a non-cerebral autoregulation model, an adjustment value that is individualized for the patient based at least in part on physiological data associated with the patient; determine a non-cerebral autoregulation status value of the patient based on the cerebral autoregulation status value and the adjustment value; and send, to an output device, a signal indicative of the non-cerebral autoregulation status value of the patient.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 is a conceptual block diagram illustrating an example autoregulation monitoring system.

FIG. 2 illustrates details of an example training system that may perform training of the non-cerebral autoregulation model shown in FIG. 1.

FIG. 3 illustrates an example deep learning architecture of the example non-cerebral autoregulation model of FIGS. 1 and 2.

FIG. 4 illustrates an example deep learning architecture of the example non-cerebral autoregulation model of FIGS. 1 and 2.

FIG. 5 illustrates an example graph that illustrates a correspondence between the cerebral autoregulation status value of a patient and the kidney autoregulation status value of the patient.

FIG. 6 is a block diagram that illustrates the autoregulation monitoring device of FIG. 1 determining a non-cerebral autoregulation status value for a patient.

FIG. 7 illustrates an example graph that illustrates an acute kidney injury threshold for a patient.

FIG. 8 is a block diagram that illustrates the autoregulation monitoring device of FIG. 1 determining an acute kidney injury threshold for a patient.

FIG. 9 illustrates an example user interface that includes autoregulation information.

FIG. 10 is a flow diagram illustrating an example method for monitoring the non-cerebral autoregulation status of a patient.

FIG. 11 is a flow diagram illustrating an example method for monitoring the non-cerebral autoregulation status of a patient.

### DETAILED DESCRIPTION

An intact autoregulation status of a subject occurs over a range of blood pressures defined between a lower limit of autoregulation (“LLA”) and an upper limit of autoregulation (“ULA”). An impaired autoregulation status occurs outside of the range of blood pressures defined between the LLA and the ULA and may occur when a patient's autoregulation process is not functioning properly. When a patient exhibits an impaired autoregulation status, the patient may experience inappropriate cerebral blood flow, which may be undesirable. For example, below a respective LLA, a drop in blood flow to a respective organ may cause ischemia and adversely affect the respective organ. Above a respective ULA, an increase in blood flow to a respective organ may cause hyperemia, which may result in swelling or edema of the respective organ. A clinician may monitor the autoregulation status of a patient, e.g., during a medical procedure, and take one or more actions to keep the patient in or bring the patient to an intact autoregulation status, such as by increasing or decreasing the patient's blood pressure.

Different organs and organ system may have different LLA and ULA values. For example, the LLA of the brain (“cerebral LLA” or “LLAc”) may be less than the LLA of the kidneys (“LLAx”) or the LLA of the gastrointestinal tract (“gut”) (“LLAG”). Similarly, the ULA of the brain (“cerebral ULA” or “ULAc”) may be greater than the ULA of the kidneys (“ULAx”) or the ULA of the gastrointestinal tract (“ULAG”). In some examples, LLAc (or ULAc) may differ from the LLAx or LLAG (or ULAx or ULAc) by greater than 5 millimeters of mercury (mmHg), such as 10 mmHg, or more. These different values reflect that organs and organ systems, such as the kidneys and the gastrointestinal tract may be adversely affected by an impaired autoregulation status before the brain is affected. Thus, outputting information about the autoregulation status of the brain as well as one or more other organs and organ systems, such as the kidneys, gastrointestinal tract, and heart, may allow a clinician to prevent a patient entering or remaining in an impaired autoregulation status for non-cerebral organs or organ systems, even when cerebral autoregulation is still intact.

A system configured to monitor an autoregulation status of a patient may be configured to determine an autoregulation status value of a non-cerebral organ of the patient based on various physiological parameters of the patient, demographic data of the patient, and/or results from laboratory analysis of the organ functions of the patient. Aspects of this disclosure describe devices, systems, and techniques for determining a non-cerebral autoregulation status, such as an LLA or a ULA for non-cerebral organs, such as kidneys. Because physiological parameters to determine autoregulation status of a non-cerebral organ may be difficult to directly measure, the described devices, systems, and techniques may determine the non-cerebral autoregulation status value based on a determined cerebral autoregulation status value.

For example, an autoregulation monitoring device may include processing circuitry configured to receive a blood pressure signal indicative of a blood pressure of the patient and an oxygen saturation signal indicative of a regional oxygen saturation of the patient. The blood pressure of the patient may be obtained by any suitable blood pressure measurement technique. In some examples, the blood pressure of the patient may include an arterial blood pressure measured using a non-invasive blood pressure measurement, such as a blood pressure derived from external cuff or photoplethysmogram, or an invasive blood pressure, such as a blood pressure derived from an intra-arterial blood pressure monitor. For example, the blood pressure value may include, or be representative of, the middle cerebral artery in the brain of the patient. The regional oxygen saturation of the patient may include any suitable regional oxygen saturation value. For example, the oxygen saturation value may include, or be representative of, an oxygen saturation at the brain of the patient.

The processing circuitry may determine a metric (e.g., a numerical value or qualitative information) indicative of the cerebral autoregulation status of the patient based on the blood pressure signal and the oxygen saturation signal. For example, a cerebral autoregulation status value may include a limit of cerebral autoregulation, such as LLAc and/or ULAc, of the patient that may be determined based on the blood pressure signal and the oxygen saturation signal. In some examples, the LLAc and/or the ULAc may be determined based on cerebral perfusion pressure. Cerebral perfusion pressure may be determined based on the blood pressure signal and intracranial pressure of the patient. In some examples, the processing circuitry may determine the LLAc and/or the ULAc based on a correlation index (COx) of the blood pressure value and oxygen saturation value. Alternatively or additionally, the processing circuitry may determine the LLAc and/or the ULAc based on other parameters or correlation coefficients.

For example, in some examples, the processing circuitry may determine the LLA and/or the ULA based on a comparison of a threshold value to a change in the blood pressure (and/or oxygen saturation) of a patient over time, e.g., determining a correlation coefficient only if the change in blood pressure (and/or oxygen saturation) over time exceeds the threshold value. In some examples, as described in commonly assigned U.S. Patent Application Publication No. 2018/0014791 naming inventors Montgomery et al. and entitled, “SYSTEMS AND METHODS OF MONITORING AUTOREGULATION,” which is hereby incorporated by reference in its entirety, the processing circuitry may process a blood pressure signal and an oxygen saturation signal to determine respective gradients of the signals (i.e., a blood pressure gradient and an oxygen saturation gradient) over a period of time and determine the patient's autoregulation status based on the respective gradients. As described in U.S. Patent Application Publication No. 2018/0014791, the processing circuitry may determine the autoregulation system of the patient may be impaired if the blood pressure gradient and the oxygen saturation gradient trend together (e.g., change in the same direction) over a period of time. In some cases, the processing circuitry may determine that the autoregulation system of the patient may be intact if the blood pressure gradient and the oxygen saturation gradient do not trend together (e.g., do not change in the same direction, such as change in different directions, or the blood pressure changes while the oxygen saturation remains generally stable) over the period of time. In some examples, the processing circuitry may execute one or more neural network algorithms trained using machine learning to associate various physiological parameters of the patient with a cerebral autoregulation status value of the patient.

The processing circuitry may further determine a non-cerebral autoregulation status (e.g., a value, or metric, such as LLA and/or ULA for the non-cerebral organ) based on a cerebral autoregulation status (e.g., value, or metric, such as a LLAc and/or a ULAc) and an adjustment value that is individualized for patient 101. In some examples, the processing circuitry may execute one or more neural network algorithms trained using machine learning to associate various physiological parameters of the patient, demographic data of the patient, and/or results from laboratory analysis of the organ functions of the patient with an adjustment value that is the difference between the cerebral autoregulation status value of the patient and the non-cerebral autoregulation status value of the patient. In some examples, the adjustment value may include a range of adjustment values, such that the non-cerebral autoregulation status value includes a range of non-cerebral autoregulation status values.

The processing circuitry may provide a signal indicative of the cerebral autoregulation status and a signal the non-cerebral autoregulation status to an output device. Output based on the signal may enable a clinician to monitor the cerebral autoregulation status and the non-cerebral autoregulation status of the patient. In some examples, a clinician may monitor the cerebral autoregulation status and the non-cerebral autoregulation status of the patient during surgery. The output device can provide, for example, a visual output, an audio output, a somatosensory output, or any combination thereof, that provides information indicative of the cerebral autoregulation status and the non-cerebral autoregulation status of the patient to the clinician. The devices, systems, and techniques of this disclosure may enable a clinician to monitor the autoregulation status of the patient and correct an impaired autoregulation status to reduce adverse effect to organ or organ systems of the patient other than or in addition to the brain of the patient. For example, during surgery an anesthesiologist may adjust therapy based on the information indicative of the cerebral autoregulation status and the non-cerebral autoregulation status to improve the autoregulation statuses, e.g., to preserve intact status. Providing the indicative of the cerebral autoregulation status and the non-cerebral autoregulation status may better help the clinician avoid organ dysfunction that might otherwise occur in non-cerebral organs even as cerebral autoregulation remains in an intact state.

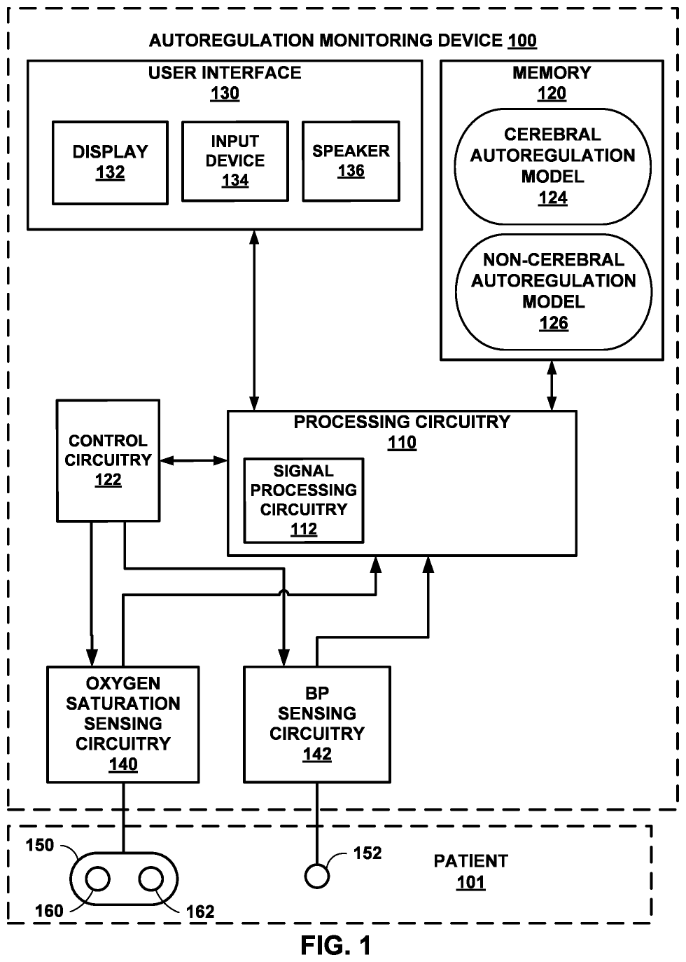

FIG. 1 is a conceptual block diagram illustrating an example autoregulation monitoring system 100. As shown in FIG. 1, autoregulation monitoring system 100 includes processing circuitry 110, memory 120, control circuitry 122, user interface 130, sensing circuitry 140 and 142, and sensing devices 150 and 152. In the example shown in FIG. 1, user interface 130 may include display 132, input device 134, and/or speaker 136, which may be any suitable audio device configured to generate and output a noise and include any suitable circuitry. In some examples, autoregulation monitoring system 100 may be configured to determine and output (e.g., for display at display 132) the cerebral autoregulation status of a patient 101, e.g., during a medical procedure or for more long-term monitoring, such as in the intensive care unit (ICU) or for fetal monitoring. A clinician may receive information regarding the cerebral autoregulation status information of a patient via user interface 130 and adjust treatment or therapy to patient 101 based on the cerebral autoregulation status information.

Processing circuitry 110, as well as other processors, processing circuitry, controllers, control circuitry, and the like, described herein, may include one or more processors. Processing circuitry 110 may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, processing circuitry 110 may include multiple components, such as any combination of one or more microprocessors, one or more DSPs, one or more ASICs, or one or more FPGAs, as well as other discrete or integrated logic circuitry, and/or analog circuitry.

Control circuitry 122 may be operatively coupled processing circuitry 110. Control circuitry 122 is configured to control an operation of sensing devices 150 and 152. In some examples, control circuitry 122 may be configured to provide timing control signals to coordinate operation of sensing devices 150 and 152. For example, sensing circuitries 140 and 142 may receive from control circuitry 122 one or more timing control signals, which may be used by sensing circuitry 140 and 142 to turn on and off respective sensing devices 150 and 152, such as to periodically collect calibration data using sensing devices 150 and 152. In some examples, processing circuitry 110 may use the timing control signals to operate synchronously with sensing circuitry 140 and 142. For example, processing circuitry 110 may synchronize the operation of an analog-to-digital converter and a demultiplexer with sensing circuitry 140 and 142 based on the timing control signals.

Memory 120 may be configured to store, for example, monitored physiological parameter values of patient 101, such as blood pressure values, oxygen saturation values, regional cerebral oxygen saturation (rSO2) values, one or more cerebral autoregulation status values, one or more non-cerebral autoregulation status values, one or more autoregulation adjustment values, physiological parameters, mean arterial pressure (MAP) values, urine flow rate values, urine oxygenation levels, and/or one or more COx values, BVS values, HVx values, or any combination thereof, and/or raw or parameterized sensor signals indicative of the aforementioned values.

Memory 120 may also be configured to store demographic data associated with patient 101, such as the weight of patient 101, the height of patient 101, the body mass index of patient 101, the sex of patient 101, the disease state of one or more diseases (e.g., renal diseases) of patient 101, as well as the results of laboratory analysis or other analysis (e.g., by sensors fluidically coupled to urine flow from or through a Foley catheter) of urine or another fluid of patient 101. In the case of urine, the analysis can include serum creatine, calcium, pH, and/or oxygen level of urine, or other relevant measures of kidney function taken at the same or at different periods in time for patient 101. The analysis of a body fluid of patient 101 is generally referred to herein as laboratory analysis, but the analysis can be automatically conducted via a sensor in a Foley catheter or otherwise in fluid communication with the body fluid.

Memory 120 may also be configured to store data such as autoregulation state values including modified and unmodified values, threshold values and rates, smoothing functions, Gaussian filters, confidence metrics, expected autoregulation functions, historical patient blood pressure value data, and/or estimates of limits of autoregulation. The threshold values and rates, smoothing functions, Gaussian filters, confidence metrics, expected autoregulation functions, and historical patient blood pressure value data may stay constant throughout the use of system 100 and across multiple patients, or these values may change over time. In some examples, data may be stored in memory 120 as one or more look-up tables or equations defining one or more associations (e.g., relationships) between stored data, such as, for example, associations between cerebral autoregulation status values and non-cerebral autoregulation status values.

In some examples, memory 120 may store program instructions, such as neural network algorithms. The program instructions may include one or more program modules that are executable by processing circuitry 110. For example, memory 120 may store cerebral autoregulation model 124, which may be a model trained via machine learning to determine a cerebral autoregulation status value of patient 101, such as the cerebral LLA and/or cerebral ULA, and non-cerebral autoregulation model 126, which may be a model trained via machine learning to determine the cerebral autoregulation status value of patient 101, such as the kidney LLA and/or kidney LLA. When executed by processing circuitry 110, such program instructions, such as program instructions of cerebral autoregulation model 124 and non-cerebral autoregulation model 126, may cause processing circuitry 110 to provide the functionality ascribed to it herein. The program instructions may be embodied in software, firmware, and/or RAMware. Memory 120 may include any one or more of volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. In other examples, memory 120 does not include cerebral autoregulation model 124 and the cerebral autoregulation status of patient 101 can be determined using other techniques.

User interface 130 may include a display 132, an input device 134, and a speaker 136. In some examples, user interface 130 may include fewer or additional components. User interface 130 is configured to present information to a user (e.g., a clinician). For example, user interface 130 and/or display 132 may include a monitor, cathode ray tube display, a flat panel display such as a liquid crystal (LCD) display, a plasma display, a light emitting diode (LED) display, and/or any other suitable display. In some examples, user interface 130 may be part of a multiparameter monitor (MPM) or other physiological signal monitor used in a clinical or other setting, a personal digital assistant, mobile phone, tablet computer, laptop computer, any other suitable computing device, or any combination thereof, with a built-in display or a separate display.

In some examples, processing circuitry 110 is configured to present, by user interface 130, such as display 132, a graphical user interface to a user. The graphical user interface may include indications of values of one or more physiological parameters of a patient, such as, for example, blood pressure values, oxygen saturation values, information about an autoregulation status (e.g., cerebral autoregulation status values and/or non-cerebral autoregulation status values), pulse rate information, respiration rate information, other patient physiological parameters, or combinations thereof via display 132. User interface 130 may also include circuitry and other components configured to generate and project an audio output to a user, such as speaker 136.

In some examples, processing circuitry 110 may also receive input signals from additional sources (not shown), such as a user. For example, processing circuitry 110 may receive from input device 134, such as a keyboard, a mouse, a touch screen, buttons, switches, a microphone, a joystick, a touch pad, or any other suitable input device or combination of input devices, an input signal. The input signal may contain information about patient 101, such as physiological parameters, treatments provided to patient 101, or the like. Additional input signals may be used by processing circuitry 110 in any of the determinations or operations it performs in accordance with processing circuitry 110.

In some examples, if processing circuitry 110 determines that the cerebral autoregulation status and/or non-cerebral autoregulation status of patient 101 is impaired, then processing circuitry 110 presents a notification indicating the impairment. The notification may include a visual, audible, tactile, or somatosensory notification (e.g., an alarm signal) indicative of the cerebral and/or non-cerebral autoregulation status of patient 101. In some examples, processing circuitry 110 and user interface 130 may be part of the same device or supported within one housing (e.g., a computer or monitor). In other examples, processing circuitry 110 and user interface 130 may be separate devices configured to communicate through a wired connection or a wireless connection (e.g., a communication interface).

Oxygen saturation sensing circuitry 140 and blood pressure sensing circuitry (collectively, sensing circuitry 140 and 142) may be configured to receive physiological signals sensed by respective sensing devices 150 and 152 and communicate the physiological signals to processing circuitry 110. Sensing devices 150 and 152 may include any sensing hardware configured to sense a physiological parameter of a patient, such as, but not limited to, one or more electrodes, optical receivers, blood pressure cuffs, or the like. The sensed physiological signals may include signals indicative of physiological parameters of patient 101, such as, but not limited to, blood pressure, regional oxygen saturation, blood volume, heart rate, and respiration. For example, sensing circuitries 140 and 142 may include, but are not limited to, blood pressure sensing circuitry, oxygen saturation sensing circuitry, regional oxygen saturation sensing circuitry, regional cerebral oxygen saturation sensing circuitry, blood volume sensing circuitry, heart rate sensing circuitry, temperature sensing circuitry, electrocardiography (ECG) sensing circuitry, electroencephalogram (EEG) sensing circuitry, or any combination thereof.

In some examples, sensing circuitry 140 and 142 and/or processing circuitry 110 may include signal processing circuitry 112 configured to perform any suitable analog conditioning of the sensed physiological signals. For example, sensing circuitries 140 and 142 may communicate to processing circuitry 110 an unaltered (e.g., raw) signal. Processing circuitry 110, e.g., signal processing circuitry 112, may be configured to modify a raw signal to a usable signal by, for example, filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. In some examples, the conditioned analog signals may be processed by an analog-to-digital converter of signal processing circuitry 112 to convert the conditioned analog signals into digital signals. In some examples, signal processing circuitry 112 may operate on the analog or digital form of the signals to separate out different components of the signals. In some examples, signal processing circuitry 112 may perform any suitable digital conditioning of the converted digital signals, such as low pass, high pass, band pass, notch, averaging, or any other suitable filtering, amplifying, performing an operation on the signal, performing any other suitable digital conditioning, or any combination thereof. In some examples, signal processing circuitry 112 may decrease the number of samples in the digital detector signals. In some examples, signal processing circuitry 112 may remove dark or ambient contributions to the received signal. Additionally or alternatively, sensing circuitries 140 and 142 may include signal processing circuitry 112 to modify one or more raw signals and communicate to processing circuitry 110 one or more modified signals.

In some examples, oxygen saturation sensing device 150 is a regional oxygen saturation sensor configured to generate an oxygen saturation signal indicative of blood oxygen saturation within the venous, arterial, and/or capillary systems within a region of patient 101. For example, oxygen saturation sensing device 150 may be configured to be placed on the skin of patient 101, such as on patient 101's forehead, to determine regional oxygen saturation of a particular tissue region, e.g., the frontal cortex or another cerebral location of patient 101. Oxygen saturation sensing device 150 may include emitter 160 and detector 162. Emitter 160 may include at least two light emitting diodes (LEDs), each configured to emit at different wavelengths of light, e.g., red or near infrared light. As used herein, the term “light” may refer to energy produced by radiative sources and may include any wavelength within one or more of the ultrasound, radio, microwave, millimeter wave, infrared, visible, ultraviolet, gamma ray or X-ray electromagnetic radiation spectra. In some examples, light drive circuitry (e.g., within sensing device 150, sensing circuitry 140, control circuitry 122, and/or processing circuitry 110) may provide a light drive signal to drive emitter 160 and to cause emitter 160 to emit light. In some examples, the LEDs of emitter 160 emit light in the range of about 600 nanometers (nm) to about 1000 nm. In a particular example, one LED of emitter 160 is configured to emit light at about 730 nm and the other LED of emitter 160 is configured to emit light at about 810 nm. Other wavelengths of light may be used in other examples.

Detector 162 may include a first detection element positioned relatively “close” (e.g., proximal) to emitter 160 and a second detection element positioned relatively “far” (e.g., distal) from emitter 160. In some examples, the first detection elements and the second detection elements may be chosen to be specifically sensitive to the chosen targeted energy spectrum of light source 160. Light intensity of multiple wavelengths may be received at both the “close” and the “far” detector 162. For example, if two wavelengths are used, the two wavelengths may be contrasted at each location and the resulting signals may be contrasted to arrive at an oxygen saturation value that pertains to additional tissue through which the light received at the “far” detector passed (tissue in addition to the tissue through which the light received by the “close” detector passed, e.g., the brain tissue), when it was transmitted through a region of a patient (e.g., a patient's cranium).

In operation, light may enter detector 162 after passing through the tissue of patient 101, including skin, bone, other shallow tissue (e.g., non-cerebral tissue and shallow cerebral tissue), and/or deep tissue (e.g., deep cerebral tissue). Detector 162 may convert the intensity of the received light into an electrical signal. The light intensity may be directly related to the absorbance and/or reflectance of light in the tissue. Surface data from the skin and skull may be subtracted out, to generate an oxygen saturation signal for the target tissues over time. Such a technique described above may be referred to as near-infrared spectroscopy (NIRS), and the oxygen saturation signal may be referred to as a NIRS signal.

Oxygen saturation sensing device 150 may provide the oxygen saturation signal (e.g., regional oxygen saturation signal) to processing circuitry 110 or to any other suitable processing device to enable evaluation of an autoregulation status of patient 101. Additional example details of determining oxygen saturation based on light signals may be found in commonly assigned U.S. Pat. No. 9,861,317, which issued on Jan. 9, 2018, and is entitled “Methods and Systems for Determining Regional Blood Oxygen Saturation.”

In operation, blood pressure sensing device 152 and oxygen saturation sensing device 150 may each be placed on the same or different parts of the body of patient 101. For example, blood pressure sensing device 152 and oxygen saturation sensing device 150 may be physically separate from each other and may be separately placed on patient 101. As another example, blood pressure sensing device 152 and oxygen saturation sensing device 150 may in some cases be part of the same sensor or supported by a single sensor housing. For example, blood pressure sensing device 152 and oxygen saturation sensing device 150 may be part of an integrated oximetry system configured to non-invasively measure blood pressure (e.g., based on time delays in a plethysmography (PPG) signal) and regional oxygen saturation. One or both of blood pressure sensing device 152 or oxygen saturation sensing device 150 may be further configured to measure other parameters, such as hemoglobin, respiratory rate, respiratory effort, heart rate, saturation pattern detection, response to stimulus such as bispectral index (BIS) or electromyography (EMG) response to electrical stimulus, or the like. While an example cerebral autoregulation monitoring system 100 is illustrated in FIG. 1, the components illustrated in FIG. 1 are not intended to be limiting. Additional or alternative components and/or implementations may be used in other examples.

Blood pressure sensing device 152 may be any sensor or device configured to generate a blood pressure signal indicative of a blood pressure of patient 101 at an acquisition site. For example, blood pressure sensing device 152 may include a blood pressure cuff configured to non-invasively monitor blood pressure, a sensor configured to noninvasively generate a PPG signal, or an arterial line for invasively monitoring blood pressure in an artery of patient 101. In some examples, the blood pressure signal may include at least a portion of a waveform of the acquisition blood pressure. In some examples, an acquisition site may include at least one of a femoral artery of patient 101, a radial artery of patient 101, a dorsalis pedis artery of patient 101, a brachial artery of patient 101, or combinations thereof. In some examples, blood pressure sensing device 152 may include a plurality of blood pressure sensing devices. For example, each blood pressure sensing device of the plurality of blood pressure sensing devices may be configured to obtain a respective blood pressure of patient 101 at a respective acquisition site of a plurality of acquisition sites. The plurality of acquisition sites may include similar or different arteries of patient 101.

In some examples, blood pressure sensing device 152 may include one or more pulse oximetry sensors. The acquisition blood pressure may be derived by processing time delays between two or more characteristic points within a single PPG signal obtained from a single pulse oximetry sensor. Additional example details of deriving blood pressure based on a comparison of time delays between certain components of a single PPG signal obtained from a single pulse oximetry sensor are described in commonly assigned U.S. Patent Application Publication No. 2009/0326386 filed Sep. 30, 2008, entitled “Systems and Methods for Non-Invasive Blood Pressure Monitoring.” In other cases, the blood pressure of patient 101 may be continuously, non-invasively monitored via multiple pulse oximetry sensors placed at multiple locations on patient 101. As described in commonly assigned U.S. Pat. No. 6,599,251, issued Jul. 29, 2003, entitled “Continuous Non-invasive Blood Pressure Monitoring Method and Apparatus,” multiple PPG signals may be obtained from the multiple pulse oximetry sensors, and the PPG signals may be compared against one another to estimate the blood pressure of patient 101.

Regardless of its form, blood pressure sensing device 152 may be configured to generate a blood pressure signal indicative of a blood pressure of patient 101 (e.g., arterial blood pressure) over time. In examples in which blood pressure sensing device 152 includes a plurality of blood pressure sensing devices, the blood pressure signal may include a plurality of blood pressure signals, each indicative of a blood pressure of patient 101 at a respective acquisition site. Blood pressure sensing device 152 may provide the blood pressure signal to sensing circuitry 142, processing circuitry 110, or to any other suitable processing device to enable evaluation of the autoregulation status of patient 101.

Processing circuitry 110 may be configured to receive a blood pressure signal generated by sensing circuitry 142 and sensing device 152 that is indicative of a blood pressure of patient 101 over a period of time and an oxygen saturation signal generated by sensing circuitry 140 and sensing device 150 that is indicative of a regional oxygen saturation of patient 101 over the period of time. The period of time over which processing circuitry 110 may be the previous 30 seconds, 60 seconds 90 seconds, 120 seconds, or any other suitable period of time.

The blood pressure signal that is indicative of a blood pressure of patient 101 over a period of time may indicate the mean arterial pressure (MAP) of patient 101, the average (i.e., mean) blood pressure in patient 101 during a single cardiac cycle. As such, in some examples, the blood pressure signal may indicate a MAP for each cardiac cycle of patient 101 during the period of time.

The regional oxygen saturation (rSO2) of patient 101 indicated by the oxygen saturation signal, e.g., generated by circuitry 140 or control circuitry 122, may be the regional oxygen saturation of the brain of patient 101. In some examples, oxygen saturation sensing device 150 may include multiple sensors that are placed on different parts of patient 101, such as a sensor placed on or near the right side of the head of patient 101, and a sensor placed on or near the left side of the head of patient 101. In this example, the oxygen saturation signal that is indicative of a regional cerebral oxygen saturation of patient 101 may include a first oxygen saturation signal from an oxygen saturation sensor placed on the right side of the head of patient 101 that is indicative of a first regional cerebral oxygen saturation of patient 101 and a second oxygen saturation signal from an oxygen saturation sensor placed on the left side of the head of patient 101 that is indicative of a second regional cerebral oxygen saturation of patient 101.

In some examples, the regional oxygen saturation (rSO2) of patient 101 indicated by the oxygen saturation signal may also be the regional oxygen saturation of the kidneys of patient 101. In some examples, oxygen saturation sensing device 150 may include one or more sensors placed on or near the kidneys of patient 101 to sense the regional renal oxygen saturation of patient 101.

Processing circuitry 110 may be configured to determine physiological data associated with patient 101. Such physiological data may include the blood pressure of patient 101 over a period of time, the regional cerebral oxygen saturation of patient 101 over the period of time, and/or a regional renal oxygen saturation of patient 101 over the period of time. The physiological data associated with patient 101 may also include physiological data derived from the blood pressure of patient 101 over a period of time, the regional cerebral oxygen saturation of patient 101 over the period of time, and/or a regional renal oxygen saturation of patient 101 over the period of time. For example, processing circuitry 110 may determine a cerebral oximetry index (COx) of patient 101 during the period of time based at least in part on a linear correlation between the blood pressure of patient 101 and the regional cerebral oxygen saturation of patient 101 during the period of time. For example, processing circuitry 110 can determine the cerebral oximetry index from the correlation between cerebral oxygen saturation in the blood (rSO2) and mean arterial pressure (MAP).

In some examples, the physiological data associated with patient 101 may include the gradient of the MAP of patient 101 during the period of time, also referred to as a window, which may be the change in the MAP of patient 101 over the period of time, the gradient of the regional cerebral oxygen saturation of patient 101 during the period of time, which may be the change in the regional cerebral oxygen saturation of patient 101 over the period of time, and/or the gradient of the regional renal oxygen saturation of patient 101 during the period of time, which may be the change in the regional renal oxygen saturation of patient 101 over the period of time.

In some examples, processing circuitry 110 is configured to receive one or more signals generated by sensing devices 150 and 152 and sensing circuitry 140 and 142. The physiological signals may include a blood pressure signal indicative of a blood pressure of patient 101 and/or one or more oxygen saturation signals indicative of a cerebral oxygen saturation of patient 101 and/or a renal oxygen saturation of patient 101. After receiving one or more signals, processing circuitry 110 may be configured to determine a correlation index (e.g., COx, HVx) or other measure of autoregulation, such as based on co-trending of blood pressure and blood oxygen saturation, (e.g., based on a comparison of blood pressure gradients and oxygen saturation gradients), based on the blood pressure signal and the one or more oxygen saturation signals. In other examples, processing circuitry 110 may determine the correlation index based on additional or alternative physiological parameters (e.g., physiological signals), such as, for example, a blood volume value or a gradients measure.

In some examples, processing circuitry 110 is configured to determine, using non-cerebral autoregulation model 126, an adjustment value and to use the adjustment value along with the cerebral autoregulation status value for patient 101 to determine a non-cerebral autoregulation status value for patient 101, such as a autoregulation status value for one or more kidneys of patient 101. In some examples, the adjustment value is a value by which the non-cerebral autoregulation status value may be estimated or approximated relative to the cerebral autoregulation status value of patient 101, and may be expressed in millimeters of mercury (mmHg). In some examples, the adjustment value may include an offset, e.g., the non-cerebral autoregulation statue value (AR_{NC}) may be determined as a function of the cerebral autoregulation status value (AR_{C}) plus (or minus) the adjustment value (AV): AR_{NC}=AR_{C}+AV. For example, a kidney autoregulation status value for patient 101, such as a kidney LLA value, may be determined as a function of the cerebral LLA value plus (or minus) an LLA adjustment value. As one non-limiting example, the LLA adjustment value may be a mean difference between the kidney LLA value and the cerebral LLA value. In examples in which the adjustment value includes a range of adjustment values, the non-cerebral autoregulation status value may include a range of non-cerebral autoregulation status values. In some examples, the adjustment value may change over time.

In some examples, processing circuitry 110 may also be configured to determine, using the non-cerebral autoregulation model 126, an acute kidney injury delta value that processing circuitry 110 may use to determine a threshold value above the cerebral autoregulation value at which patient 101 may enter a blood pressure zone associated with an increased risk of developing acute kidney injury (AKI). AKI is a complication that may occur after certain medical procedures, such as surgeries that are lengthy and involve significant blood loss or fluid shifts. For example, a surgery patient's body may alter where their blood is directed to, which may lead to hypoxia of a kidney. A cause of surgery-associated AKI is hypoxia of the kidneys, which may cause an ischemia reperfusion injury to a kidney of the patient. This ischemia reperfusion injury may cause degradation of renal function of the patient.

Using the AKI delta value and a determined cerebral autoregulation status of patient 101, processing circuitry 110 can determine when a blood pressure of patient 101 is indicative of an increased risk of developing AKI, which may help a clinician intervene prior to the patient developing AKI. That is, processing circuitry 110 can determine when patient 101 may be at risk of developing AKI based on a cerebral autoregulation status. Example interventions that a clinician can take include, for example, initiating or making changes to hemodynamic management (e.g., blood pressure management, fluid management, blood transfusions, etc.), make changes to cardiopulmonary bypass machine settings, or avoid providing nephrotoxic drugs. Post operatively, a clinician may intervene with a Kidney Disease: Improving Global Outcomes (KDIGO) bundle or an AKI care bundle. The devices may be communicatively coupled to a plurality of sensors (e.g., two or more sensors) configured to sense different parameters of a fluid of interest, such as urine in the case of kidney function monitoring.

As described in further detail throughout this disclosure, non-cerebral autoregulation model 126 may include a neural network algorithm trained via machine learning to determine the adjustment value. Processing circuitry 110 may input, into autoregulation model 126, physiological data associated with patient 101. In some examples, such physiological data may include the blood pressure of patient 101 during a period of time, the regional cerebral oxygen saturation of patient 101 during the period of time, and/or the regional renal oxygen saturation of patient 101 during the period of time. The physiological data associated with patient 101 that is inputted into autoregulation model 126 may also include physiological data derived from one or more of: the blood pressure of patient 101 during the period of time, the regional cerebral oxygen saturation of patient 101 during the period of time, and/or the regional renal oxygen saturation of patient 101 during the period of time, such as the gradient of the blood pressure (e.g., MAP) of patient 101 during the period of time, the gradient of the regional cerebral oxygen saturation of patient 101 during the period of time, and/or the COx of patient 101 during the period of time.

In some examples, if processing circuitry 110 receives an oxygen saturation signal in the form of a NIRS signal, then processing circuitry 110 may include the raw NIRS signal indicative of the regional cerebral oxygen saturation of patient 101 in the physiological data associated with patient 101 that is inputted into non-cerebral autoregulation model 126. In some examples, if processing circuitry 110 receives an oxygen saturation signal that includes a first oxygen saturation signal from an oxygen saturation sensor placed on the right side of the head of patient 101 that is indicative of a first regional cerebral oxygen saturation of patient 101 and a second oxygen saturation signal from an oxygen saturation sensor placed on the left side of the head that is indicative of a second regional cerebral oxygen saturation of patient 101, then processing circuitry 110 may include both the first and second regional cerebral oxygen saturation signals in the physiological data associated with patient 101 that is inputted into non-cerebral autoregulation model 126. For example, processing circuitry 110 may input the two separate values into non-cerebral autoregulation model 126 or may input an average of the two values into non-cerebral autoregulation model 126. In some examples, processing circuitry 110 may also include the blood oxygen saturation (SpO²) of patient 101 in the physiological data associated with patient 101 that is inputted into non-cerebral autoregulation model 126.

In some examples, processing circuitry 110 may also input, into non-cerebral autoregulation model 126, additional physiological data associated patient 101 and determine the cerebral autoregulation status of patient 101 based on the additional physiological data. For example, such additional physiological data may include morphology characteristics associated with a blood pressure signal generated by circuitry 140 or 122 and indicative of the blood pressure of patient 101 during the period of time and/or morphology characteristics associated with a regional oxygen saturation signal generated by a circuitry 140 or 122 and indicative of the regional oxygen saturation of patient 101 during the period of time. The additional physiological data may also include blood pressures of patient 101 during the period of time other than the MAP, such as the systolic blood pressure or diastolic blood pressure of patient 101 during the period of time. In some examples, the additional physiological data may include a bypass flag indicative that patient 101 was undergoing a cardiopulmonary bypass procedure during the period of time, which may impact the blood pressure values of patient 101 during the time period, urine flow rate of patient 101, and/or urine oxygenation level of patient 101.

In some examples, processing circuitry 110 may also input, into non-cerebral autoregulation model 126, demographics data of patient 101, such as one or more of the following: the patient's age, the patient's weight, the patient's height, the patient's body mass index, the patient's sex, the disease state of patient 101's non-cerebral organs, such as the renal disease state of patient 101's kidneys, information regarding patient 101's diet and lifestyle (e.g., whether patient 101 is a smoker), or the like. In some examples, processing circuitry 110 may also input, into non-cerebral autoregulation model 126, results of laboratory analysis for patient 101's body fluid of interest, such as urine in the case of monitoring for acute kidney injury. Example parameters of the body fluid of interest that can be monitored include serum creatine, calcium, pH, oxygen level, or other relevant measures of kidney function for patient 101, which may be taken at the same or different periods in time.

Processing circuitry 110 may be configured to execute non-cerebral autoregulation model 126 to output, based on the information inputted into non-cerebral autoregulation model 126, an adjustment value. Processing circuitry 110 may also be configured to execute non-cerebral autoregulation model 126 to output, based on the information inputted into non-cerebral autoregulation model 126, a threshold delta value above the cerebral autoregulation status value at which patient 101 may enter a blood pressure zone associated with an increased risk of developing AKI. Such a threshold delta value is referred to herein as an AKI threshold delta value, which, when added to the cerebral autoregulation status value (e.g., LLA or ULA), results in an AKI threshold value indicative of a blood pressure threshold at which patient 101 may enter a blood pressure zone associated with an increased risk of developing AKI.

Non-cerebral autoregulation model 126 includes a neural network algorithm trained via machine learning to take a plurality of data, such as the physiological data associated with patient 101 and/or the demographic data associated with patient 101, as inputs to determine an adjustment value, which may be a difference (e.g., mean difference) between the cerebral autoregulation status value for patient 101 and the non-cerebral autoregulation status value for patient 101, and, in some examples, to determine the AKI threshold delta value. In some examples, non-cerebral autoregulation model 126 is a neural network algorithm trained via machine learning to take a plurality of signals, including physiological data of patient 101 and/or demographic data of patient 101, as inputs to determine an adjustment value, which may be a difference (e.g., mean difference) between the cerebral autoregulation status value for patient 101 and the non-cerebral autoregulation status value for patient 101, and/or to determine an AKI threshold delta value.

A neural network algorithm, or artificial neural network, may include a trainable or adaptive algorithm utilizing nodes that define rules. For example, a respective node of a plurality of nodes may utilize a function, such as a non-linear function or if-then rules, to generate an output based on an input. A respective node of the plurality of nodes may be connected to one or more different nodes of the plurality of nodes along an edge, such that the output of the respective node includes the input of the different node. The functions may include parameters that may be determined or adjusted using a training set of inputs and desired outputs, such as, for example, a predetermined association between a plurality of signals or values, such as a blood pressure signal or blood pressure value(s) from patient 101 or a population of patients and an oxygen saturation signal or oxygen saturation value(s) of patient 101 or a population of patients measured contemporaneously with the blood pressure signal, along with a learning rule, such as a back-propagation learning rule. The back-propagation learning rule may utilize one or more error measurement comparing the desired output to the output produced by the neural network algorithm to train the neural network algorithm by varying the parameters to minimize the one or more error measurements.

An example neural network includes a plurality of nodes, at least some of the nodes having node parameters. An input including physiological data of patient 101 and/or demographic data of patient 101 may be provided (input) to a first node of the neural network algorithm. In some examples, the input may include a plurality of inputs, each input into a respective node. The first node may include a function configured to determine an output based on the input and one or more adjustable node parameters. In some examples, the neural network may include a propagation function configured to determine an input to a subsequent node based on the output of a preceding node and a bias value. In some examples, a learning rule may be configured to modify one or more node parameters to produce a favored output. For example, the favored output may be constrained by one or more threshold values and/or to minimize one or more error measurements. The favored output may include an output of a single node, a set of nodes, or the plurality of nodes.

The neural network algorithm may iteratively modify the node parameters until the output includes the favored output. In this way, processing circuitry 110 may be configured to iteratively evaluating outputs of the neural network algorithm and iteratively modifying at least one of the node parameters based on the evaluation of the outputs of the neural network algorithm to determine an adjustment value for a patient, such as patient 101 and/or an AKI threshold delta value, based on the modified neural network algorithm.

In accordance with aspects of the present disclosure, processing circuitry 110 may be configured to determine a non-cerebral autoregulation status value for patient 101 based on the cerebral autoregulation status value of patient 101 and an adjustment value that is individualized for patient 101. The adjustment value may define a characteristic difference, such as, for example, a mean difference, a maximum difference, a minimum difference, a percentile difference, of the like, between a cerebral autoregulation status value and a non-cerebral autoregulation status value. Because it may be challenging to directly and non-invasively sense physiological parameters of the non-cerebral organ due to the placement of the non-cerebral organ in the body, in some examples, using the adjustment value determined by any of the ways described herein may provide relatively accurate approximation of non-cerebral autoregulation status value relative to the cerebral autoregulation status value. Because the adjustment value is determined based on patient 101's data, such as patient 101's physiological data and/or demographic data, the non-cerebral autoregulation status value determined for patient 101 is an individualized estimate of the non-cerebral autoregulation status value of patient 101.

In the event that processing circuitry 110 is unable to determine an adjustment value using non-cerebral autoregulation model 126, processing circuitry 110 may be configured to set a minimum adjustment value, such as 1 mmHg, 3 mmHg, 5 mmHg, and the like that processing circuitry 110 may use as the adjustment value to determine the non-cerebral autoregulation status value of patient 101. In some examples, processing circuitry 110 may be configured to determine such a minimum adjustment value based on a previous analysis from a cohort of patients. In some examples, processing circuitry 110 may be configured to determine such a minimum adjustment value based on the cerebral autoregulation status value of patient 101, such as by increasing or decreasing the minimum adjustment value based on the cerebral autoregulation status value of patient 101. In some examples, processing circuitry 110 may be configured to determine such a minimum adjustment value by setting the AKI threshold delta value as the minimum adjustment value.

Processing circuitry 110 may be configured to determine the cerebral autoregulation status value of patient 101 via any suitable technique, such as by executing cerebral autoregulation model 124 which may receive, as inputs, a blood pressure signal and a regional cerebral oxygen saturation signal to determine the cerebral autoregulation status value of patient 101, such as the cerebral LLA of patient 101 and/or the cerebral ULA of patient 101. In some, but not all, examples, cerebral autoregulation model 124 is a neural network algorithm, similar to non-cerebral autoregulation model 126, trained via machine learning to take a plurality of signals, including a blood pressure signal indicative of the blood pressure of patient 101 and an oxygen saturation signal indicative of the regional oxygen saturation of patient 101 as inputs to determine the cerebral autoregulation status value of patient 101. Other techniques that processing circuitry 110 can implement to determine the cerebral autoregulation status value are described above.

After determining the cerebral autoregulation status value of patient 101 and the adjustment value, processing circuitry 110 may determine a non-cerebral autoregulation status value of patient 101 based on the cerebral autoregulation status value of patient 101 and the adjustment value. For example, if the cerebral autoregulation status value of patient 101 is a cerebral LLA of patient 101, then processing circuitry 110 may determine non-cerebral autoregulation status value of patient 101, such as a kidney LLA of patient 101, as the sum of the cerebral LLA and the adjustment value. In another example, if the cerebral autoregulation status value of patient 101 is a cerebral ULA of patient 101, then processing circuitry 110 may determine non-cerebral autoregulation status value of patient 101, such as a kidney ULA of patient 101, as the sum of the cerebral ULA and the adjustment value.

In some examples, processing circuitry 110 is also configured to determine the AKI threshold value of patient 101 based on the cerebral autoregulation status value of patient 101 and the AKI threshold delta value. For example, processing circuitry 110 may be configured to determine the AKI threshold value as the sum of the cerebral autoregulation status value of patient 101 and the AKI threshold delta value.

Once processing circuitry 110 has determined the non-cerebral autoregulation status of patient 101 and/or the AKI threshold value of patient 101, processing circuitry 110 may generate and output information indicative of the non-cerebral autoregulation status value of patient 101 to an output device, e.g., user interface 130. In some examples, processing circuitry 110 can also generate and output information indicative of the cerebral autoregulation status value of patient 101 and/or the AKI threshold value of patient 101 to the output device. In some examples, the information may enable user interface 130, for example, display 132, speaker 136, and/or separate display(s) (not shown), to present a graphical user interface that includes information indicative of an autoregulation status of patient 101, such as the cerebral autoregulation status value, the non-cerebral autoregulation status value, the AKI threshold value, an indication of an impaired autoregulation state of the brain and/or the non-cerebral organ, and/or an indication of an increased risk of patient 101 developing AKI determined based on the cerebral autoregulation status and the AKI threshold value. In some examples, the indication of autoregulation status may include text, colors, and/or audio presented to a user.

Processing circuitry 110 may be further configured to present an indication of one or more non-cerebral autoregulation status values, one or more cerebral autoregulation status values, one or more limits of autoregulation (e.g., LLAc, LLAK, LLAG, ULAc, ULAx, and/or ULAG), blood pressure(s), oxygen saturation(s), or the like, on the graphical user interface. In addition to or instead of the graphical user interface, processor circuitry 110 may be configured to generate and present information indicative of a determined autoregulation status of patient 101 via speaker 136. For example, in response to detecting an impaired autoregulation state of patient 101, processing circuitry 110 may generate an audible alert via speaker 136.

In some examples, autoregulation monitoring system 100, e.g., processing circuitry 110 or user interface 130, may include a communication interface to enable autoregulation monitoring system 100 to exchange information with external devices. The communication interface may include any suitable hardware, software, or both, which may allow cerebral autoregulation monitoring system 100 to communicate with electronic circuitry, a device, a network, a server or other workstations, a display, or any combination thereof. For example, processing circuitry 110 may receive blood pressure values, oxygen saturation values, or predetermined data, such as one or more of a predetermined adjustment value for determining a non-cerebral autoregulation status of patient 101, a predetermined AKI threshold value, predetermined cerebral autoregulation status values, predetermined non-cerebral autoregulation status value, or predetermined adjustment values from an external device via the communication interface.

The components of autoregulation monitoring system 100 that are illustrated and described as separate components are illustrated and described as such for illustrative purposes only. In some examples the functionality of some of the components may be combined in a single component. For example, the functionality of processing circuitry 110 and control circuitry 122 may be combined in a single processor system. Additionally, in some examples the functionality of some of the components of autoregulation monitoring system 100 illustrated and described herein may be divided over multiple components. For example, some or all of the functionality of control circuitry 122 may be performed in processing circuitry 110, or sensing circuitry 140 and 142. In other examples, the functionality of one or more of the components may be performed in a different order or may not be required.

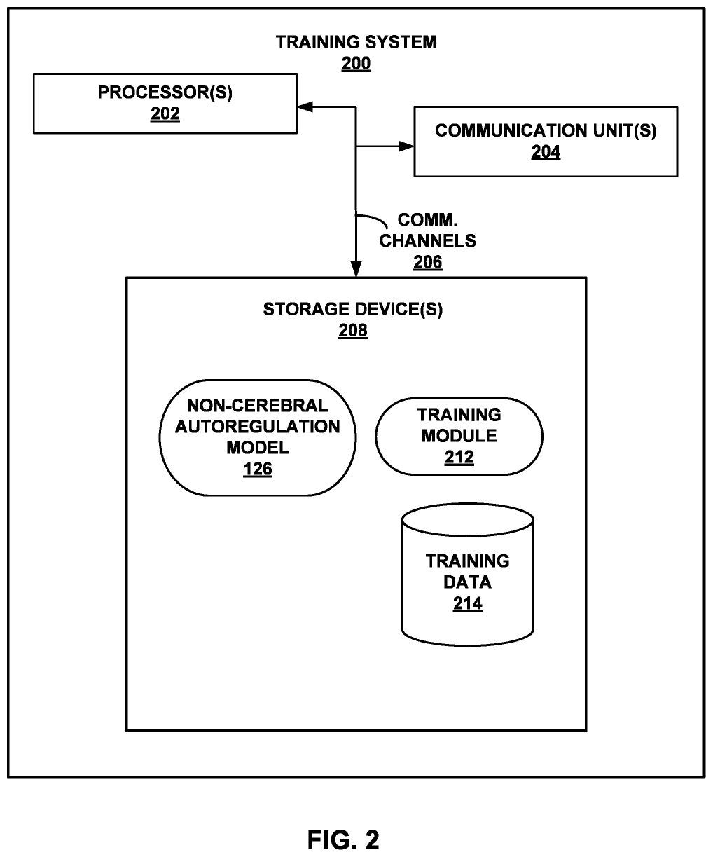

FIG. 2 illustrates details of an example training system 200 that may perform training of non-cerebral autoregulation model 126 shown in FIG. 1. FIG. 2 illustrates only one particular example of training system 200, and many other example devices having more, fewer, or different components may also be configurable to perform operations in accordance with techniques of the present disclosure.

While displayed as part of a single device in the example of FIG. 2, components of training system 200 may, in some examples, be located within and/or be a part of different devices. For instance, in some examples, training system 200 may represent a “cloud” computing system. Thus, in these examples, the modules illustrated in FIG. 2 may span across multiple computing devices. In some examples, training system 200 may represent one of a plurality of servers making up a server cluster for a “cloud” computing system. In other examples, training system 200 may be an example of the autoregulation device 100 shown in FIG. 1.

As shown in the example of FIG. 2, training system 200 includes one or more processors 202, one or more communications units 204, and one or more storage devices 208. Storage devices 208 further include non-cerebral autoregulation model 126, training module 212, and training data 214. Each of components 202, 204, and 208 may be interconnected (physically, communicatively, and/or operatively) for inter-component communications. In the example of FIG. 2, components 202, 204, and 208 may be coupled by one or more communications channels 206. In some examples, communications channels 206 may include a system bus, network connection, inter-process communication data structure, or any other channel for communicating data. Non-cerebral autoregulation model 126, training module 212, and training data 214 may also communicate information with one another as well as with other components in training system 200.

In the example of FIG. 2, one or more processors 202 (each including processing circuitry) may implement functionality and/or execute instructions within training system 200. For example, one or more processors 202 may receive and execute instructions stored by storage devices 208 that execute the functionality of training module 212. These instructions executed by one or more processors 202 may cause training system 200 to store information within storage devices 208 during execution. One or more processors 202 may execute instructions of training module 212 to train non-cerebral autoregulation model 126 using training data 214. That is, training module 212 may be operable by one or more processors 202 to perform various actions or functions of training system 200 described herein.

In the example of FIG. 2, one or more communication units 204 may be operable to communicate with external devices (e.g., system 100 of FIG. 1) via one or more networks by transmitting and/or receiving network signals on the one or more networks. For example, training system 200 may use communication units 204 to transmit and/or receive radio signals on a radio network such as a cellular radio network. Likewise, communication units 204 may transmit and/or receive satellite signals on a satellite network such as a global positioning system (GPS) network. Examples of communication units 204 include a network interface card (e.g. such as an Ethernet card), an optical transceiver, a radio frequency transceiver, or any other type of device that can send and/or receive information. Other examples of communication units 204 may include Near-Field Communications (NFC) units, Bluetooth® radios, short wave radios, cellular data radios, wireless network (e.g., Wi-Fi®) radios, as well as universal serial bus (USB) controllers.

One or more storage devices 208 may be operable, in the example of FIG. 2, to store information for processing during operation of training system 200. In some examples, storage devices 208 may represent temporary memory, meaning that a primary purpose of storage devices 208 is not long-term storage. For instance, storage devices 208 of training system 200 may be volatile memory, configured for short-term storage of information, and therefore not retain stored contents if powered off. Examples of volatile memories include random access memories (RAM), dynamic random access memories (DRAM), static random access memories (SRAM), and other forms of volatile memories known in the art.

Storage devices 208, in some examples, also represent one or more computer-readable storage media. That is, storage devices 208 may be configured to store larger amounts of information than a temporary memory. For instance, storage devices 46 may include non-volatile memory that retains information through power on/off cycles. Examples of non-volatile memories include magnetic hard discs, optical discs, floppy discs, flash memories, or forms of electrically programmable memories (EPROM) or electrically erasable and programmable (EEPROM) memories. In any case, storage devices 208 may, in the example of FIG. 2, store program instructions and/or data associated with non-cerebral autoregulation model 126, training module 212, and training data 214.

Training system 200 may, in the example of FIG. 2, execute training module 212 to train non-cerebral autoregulation model 126 using training data 214 to more accurately and/or more quickly determine an adjustment value that may be used in combination with the cerebral autoregulation status value of patient to determine the non-cerebral autoregulation status value of an organ, such as the kidneys, of the patient by training non-cerebral autoregulation model 126 to associate one or more features with an autoregulation adjustment value. In some examples, training system 200 may also execute training module 212 to train non-cerebral autoregulation model 126 using training data 214 to more accurately and/or more quickly determine an AKI threshold delta value that may be used in combination with the cerebral autoregulation status value of patient to determine whether the blood pressure of the patient may be entering an undesirable blood pressure, such as a blood pressure that is associated with an increased risk of developing AKI. Non-cerebral autoregulation model 126 may include a deep learning architecture such as a recurrent neural network, convolutional neural network, and the like that includes multiple layers to progressively extract higher level features from inputs to non-cerebral autoregulation model 126.

In some examples, training module 212 trains non-cerebral autoregulation model 126 to use, as inputs, physiological features of a patient and/or demographic data of the patient and to determine, based on the physiological features of the patient and/or demographics data of the patient, an autoregulation adjustment value, so that a non-cerebral autoregulation status value for the patient can be determined as a function of the autoregulation adjustment value and a cerebral autoregulation status value for the patient. In some examples, training module 212 trains non-cerebral autoregulation model 126 to use, as inputs, physiological features of a patient and/or demographics data of the patient and to determine, based on the physiological features of the patient and/or demographics data of the patient, an AKI threshold delta value.

The physiological features of a patient that non-cerebral autoregulation model 126 is trained to use as inputs may include one or more of: a blood pressure of a patient, such as the MAP of the patient, over a period of time, such as 30 seconds 60 seconds, 90 seconds, 120 seconds, and the like, a regional cerebral oxygen saturation of the patient over the period of time, a regional renal oxygen saturation of the patient over the period of time, a gradient of the blood pressure of the patient over the period of time, a gradient of the regional cerebral oxygen saturation of the patient over the period of time, a gradient of the regional renal oxygen saturation of the patient over the period of time, a cerebral oxygenation index (COx) of the patient over the period of time, a bypass flag indicating that the patient was undergoing a cardiopulmonary bypass procedure during the period of time, a first one or more morphology characteristics of the blood pressure of the patient during the period of time, a second one or more morphology characteristics of the regional cerebral oxygen saturations during the period of time, a urine flow rate of the patient, or urine oxygenation levels of the patient.

Training module 212 trains non-cerebral autoregulation model 126 to use, as inputs, physiological data such as the MAP, the regional cerebral oxygen saturation, and the regional non-cerebral oxygen saturation of a patient during a period of time to determine, based on the inputs during the period of time, an adjustment value and/or an AKI threshold delta value immediately following the period of time. Thus, if the period of time is 30 seconds, then training module 212 may train non-cerebral autoregulation model 126 to determine, based on such inputs over a 30 second time period, an adjustment value and/or an AKI threshold delta value immediately following the 30 second time period. In this way, non-cerebral autoregulation model 126 is able to determine an adjustment value and/or an AKI threshold delta value for a patient based on the actual sensed patient parameters during a medical procedure rather than setting a predetermined adjustment value and/or a predetermined AKI threshold delta value for a patient for the duration of a medical procedure, thereby enabling autoregulation monitoring system 100 to determine, for a patient during a medical procedure, a non-cerebral autoregulation status value and/or an AKI threshold value that is more accurate and more responsive to the status of the patient.

The demographic data of a patient that non-cerebral autoregulation model 126 is trained to use as inputs may include one or more of: the age of the patient, the sex of the patient, the height of the patient, the weight of the patient, the disease state of one or more non-cerebral organs of the patient, such as the disease state of the kidneys of the patient, and the like. In some examples, training module 212 may also train non-cerebral autoregulation model 126 to use, as inputs, laboratory analysis of one or more aspects, such as a body fluid, of patient 101, such as laboratory analysis for serum creatine levels, calcium levels, pH, or other relevant measures of kidney function of the patient, which may be taken at the same or different periods in time, and to determine, based on the laboratory analysis of the patient, an adjustment value and/or an AKI threshold delta value for the patient.

For example, training module 212 may train non-cerebral autoregulation model 126 by providing data over hours, days, and the like, and may train non-cerebral autoregulation model 126 by providing a truth label to label points in time in the data to indicate, for example, the adjustment value and/or an AKI threshold delta value. For example, if training module 212 is training non-cerebral autoregulation model 126 to determine, based on the inputs during a period of time of 30 seconds, an adjustment value and/or or an AKI threshold delta value immediately following the period of time, then training module 212 may label the data at 30 seconds to indicate an adjustment value and/or or an AKI threshold delta value associated with the data (e.g., the physiological data, the demographic data, and/or the laboratory analysis) from time 0 to 30 seconds, label the data at 31 seconds to indicate an adjustment value and/or or an AKI threshold delta value associated with the data from time 1 to 31 seconds, label the data at 32 seconds to indicate an adjustment value and/or or an AKI threshold delta value associated with the data from time 2 to 32 seconds, and the like.

In some examples, training module 212 may train non-cerebral autoregulation model 126 by subtracting the mean value of the MAP from the MAP of the patient, by subtracting the mean regional cerebral oxygen saturation from the regional cerebral oxygen saturation of the patient, and by subtracting the mean regional non-cerebral oxygen saturation from the regional non-cerebral oxygen saturation of the patient. For example, for the time period from 0 to 30 seconds, training module 212 may subtract the mean value of the MAP over the entire training data from the MAP during the period of time from 0 to 30 seconds, may subtract the mean value of the regional cerebral oxygen saturation over the entire training data from the regional cerebral oxygen saturation during the period of time from 0 to 30 seconds, and may subtract the mean value of the regional non-cerebral oxygen saturation over the entire training data from the regional non-cerebral oxygen saturation during the period of time from 0 to 30 seconds and may use the resulting values as inputs to train non-cerebral autoregulation model 126.

Subtracting the mean value of the MAP from the MAP of the patient, subtracting the mean regional cerebral oxygen saturation from the regional cerebral oxygen saturation of the patient, and subtracting the mean regional non-cerebral oxygen saturation from the regional non-cerebral oxygen saturation of patient 101 may help to prevent the trained neural network in non-cerebral autoregulation model 126 from overfitting based on the absolute value of the MAP. Instead, training module 212 may train non-cerebral autoregulation model 126 to determine an adjustment value and/or an AKI threshold delta value of patients based on the relationship between trends in the MAP, trends in the regional cerebral oxygen saturation, and/or trends in the regional non-cerebral oxygen saturation during the particular time period.

As described above, in addition to the MAP, the regional cerebral oxygen saturation, and the regional non-cerebral oxygen saturation, training module 212 trains non-cerebral autoregulation model 126 to use, as inputs, data in each time period such as, but not limited to, one more of: the gradient of the MAP during the time period (e.g., as a time series), the gradient of the regional cerebral oxygen saturation during the time period (e.g., as a time series), the gradient of the regional non-cerebral oxygen saturation during the time period (e.g., as a time series), the cerebral oximetry index during the time period, a flag that indicates whether patient 101 is undergoing a medical procedure that may impact blood pressure values (e.g., cardiopulmonary bypass procedure) during the time period, the raw or parameterized NIRS signal indicative of the regional cerebral and/or non-cerebral oxygen saturation during the time period, oxygen saturation signals from one or more oxygen saturation sensors, morphology characteristics associated with a blood pressure signal indicative of sensed blood pressure during the time period, morphology characteristics associated with a regional oxygen saturation signal indicative of sensed regional cerebral oxygen saturation during the time period, and/or morphology characteristics associated with a regional oxygen saturation signal indicative of sensed regional non-cerebral oxygen saturation during the time period, hemodynamic-related signals such as the systolic blood pressure or diastolic blood pressure during the time period, urine flow rate, urine oxygenation level, demographic data associated with patients, results from laboratory analysis of measures of kidney function, and the like.

In some examples, training module 212 trains non-cerebral autoregulation model 126 to also use, as inputs, the blood oxygen saturation (SpO2) of patients. Using the blood oxygen saturation of patients to train non-cerebral autoregulation model 126 to cope with changes in regional cerebral oxygen saturation and/or regional non-cerebral oxygen saturation due to changes in the blood oxygen saturation unrelated to cerebral blood flow. For example, training module 212 may train non-cerebral autoregulation model 126 to learn that changes in regional non-cerebral oxygen saturation of an organ due to changes in the blood oxygen saturation unrelated to the non-cerebral blood flow of the organ is not necessarily a sign of a change in autoregulation status of an organ of a patient.

In some examples, training data 214 used to train non-cerebral autoregulation model 126 includes data from only patient 101 and from no other subjects. Thus, training data 214 may include physiological data measured from patient 101 at a plurality of different time periods, demographic data of patient 101, and/or results from laboratory analysis of measures of patient 101's organ function (e.g., kidney function), and associations between such data and a difference between the cerebral autoregulation status value and the non-cerebral autoregulation status value (e.g., kidney autoregulation status value) of patient 101 at each of the plurality of different time periods. The training data 214 may also include physiological data measured from patient 101 at a plurality of different time periods, demographic data of patient 101, and/or results from laboratory analysis of measures of patient 101's organ function (e.g., kidney function), and associations between such data and a difference between the cerebral autoregulation status value and the AKI threshold value of patient 101 at each of the plurality of different time periods.

In other examples, training data 214 may include data from a population of patients that may or may not include patient 101. Thus, training data 214 may include physiological data measured from a plurality of patients, demographic data of the plurality of patients, and results from laboratory analysis of measures of each of the plurality of patients' organ function (e.g., kidney function). The physiological data may include the blood pressure of each of the plurality of patients, such as the MAP of each of the plurality of patients, over a period of time, such as 30 seconds 60 seconds, 90 seconds, 120 seconds, and the like, a regional cerebral oxygen saturation of each of the plurality of patients over the period of time, a regional non-cerebral (e.g., renal) oxygen saturation of each of the plurality of patients over the period of time, a gradient of the blood pressure of each of the plurality of patients over the period of time, a gradient of the regional cerebral oxygen saturation of each of the plurality of patients over the period of time, a gradient of the regional renal oxygen saturation of each of the plurality of patients over the period of time, a cerebral oxygenation index (COx) of each of the plurality of patients over the period of time, a bypass flag for each of the plurality of patients indicating whether each of the plurality of patients was undergoing a cardiopulmonary bypass procedure during the period of time, a first one or more morphology characteristics of blood pressure signals of each of the plurality of patients during the period of time, a second one or more morphology characteristics of regional cerebral oxygen saturation signals during the period of time each of the plurality of patients, a third one or more morphology characteristics of the regional non-cerebral oxygen saturations during the period of time each of the plurality of patients a urine flow rate of each of the plurality of patients, and/or urine oxygenation levels of each of the plurality of patients.

Training data 214 may also include demographic data of each of the plurality of patients, such as the age of each of the plurality of patients, the sex of each of the plurality of patients, the height of each of the plurality of patients, the weight of each of the plurality of patients, the disease state of one or more non-cerebral organs of each of the plurality of patients, such as the disease state of the kidneys of each of the plurality of patients, and the like. In some examples, training data 214 may also include laboratory analysis of each of the plurality of patients, such as laboratory analysis for serum creatine levels, calcium levels, pH, or oxygen level of urine of the respective patient, or other relevant measures of kidney function of each of the plurality of patients, which may be taken at the same or different periods in time.

In some examples, training data 214 may also include data described above that are collected from patients who have suffered from an acute kidney injury. Such data may be collected during outcome-based training and/or clinical trials, such as clinical trials during which data regarding the relationship between blood pressure levels of patients and the kidney LLAs of patients and the relationship between patients developing AKIs and the patients' blood pressures and kidney LLAs.

Training data 214 may include associations between the data of each of the plurality of patients with a difference between the cerebral autoregulation status value and the non-cerebral autoregulation status value (e.g., kidney autoregulation status value) of patient 101 at each of the plurality of different time periods. Training data 214 may also include associations between the data of each of the plurality of patients with a difference between the cerebral autoregulation status value and the AKI threshold value of patient 101 at each of the plurality of different time periods, thereby including associations between the data of each of the plurality of patients with a degree of AKI caused in the patient.

In some examples, once training module 212 has trained non-cerebral autoregulation model 126 using training data 214, training module 212 may test non-cerebral autoregulation model 126 by using a set of test data not yet encountered by using non-cerebral autoregulation model 126 to determine how closely the output of non-cerebral autoregulation model 126 based on the test data matches the expected adjustment value and/or expected AKI threshold delta value of the test data. In this way, training module 212 may evaluate and further refine non-cerebral autoregulation model 126.

When training module 212 has completed training of non-cerebral autoregulation model 126, non-cerebral autoregulation model 126 can be installed, uploaded, or otherwise transferred to autoregulation monitoring system 100. In some examples, training module 212 may upload or otherwise transfer a copy of non-cerebral autoregulation model 126 to another server or to the cloud, and autoregulation monitoring system 100 may non-cerebral autoregulation model 126 via a network such as the Internet, a virtual private network, a local area network, and the like.

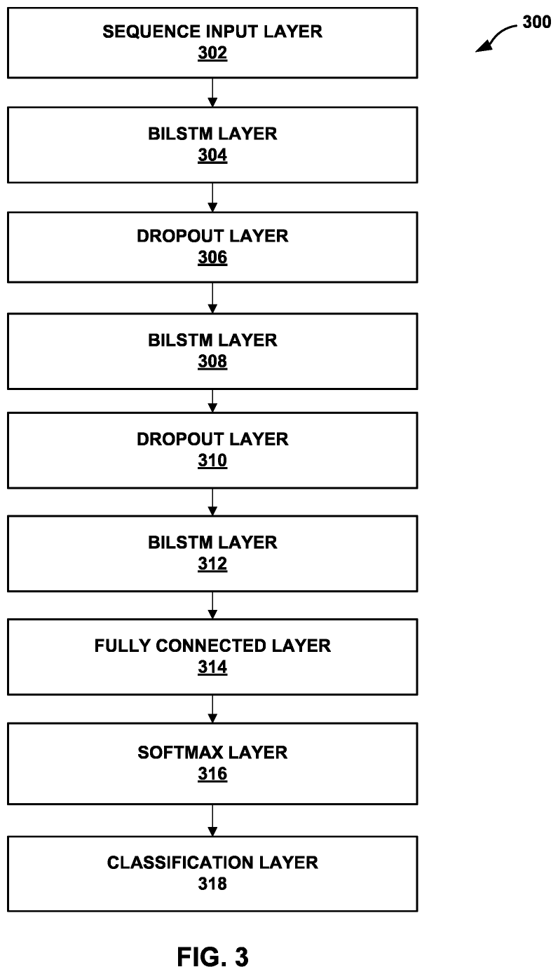

FIG. 3 illustrates an example deep learning architecture 300 of the example non-cerebral autoregulation model 126 of FIGS. 1 and 2. While deep learning architecture 300 is illustrated in FIG. 3 as being a long short-term memory (LSTM) deep learning architecture that is used to train a LSTM model, any other deep learning architectures may equally be suitable for training non-cerebral autoregulation model 126.

As shown in FIG. 3, deep learning architecture 300 may include sequence input layer 302, bidirectional long short-term memory (BiLSTM) layer 304, dropout layer 306, BiLSTM layer 308, dropout layer 310, BiLSTM layer 312, fully connected layer 314, softmax layer 316 and classification layer 318. Sequence input layer 302 may be connected to BiLSTM layer 304. BiLSTM layer 304 may have 16 hidden units and may be connected to dropout layer 306. Dropout layer 306 may have a dropout ratio of 0.01 and may be connected to BiLSTM layer 308. BiLSTM layer 308 may have 8 hidden units and may be connected to dropout layer 310. Dropout layer 310 may have a dropout ratio of 0.01 and may be connected to BiLSTM layer 312, BiLSTM layer 312 may have 4 hidden units and may be connected to fully connected layer 314. Fully connected layer 314 may be connected to softmax layer 316. Softmax layer 316 may be connected to classification layer 318.

A sequence input layer such as sequence input layer 302 inputs sequence data to a neural network. Thus, sequence input layer 302 receives features that are used to train deep learning architecture 300, such as one or more physiological features of one or more patients,

A dropout layer such as dropout layers 306 and 310 randomly sets input elements to zero with a given probability. By randomly setting input elements to zero, a dropout layer may enable elements to be ignored during the training phase. Selectively ignoring elements during the training phase may prevent over-fitting of training data.

A BiLSTM layer such as BiLSTM layers 304, 308, and 312 learns bidirectional long-term dependencies between time steps of time series or sequence data. These dependencies may be useful for the network to learn from a complete time series at each time step.

A fully connected layer such as fully connected layer 314 multiplies the input (e.g., from BiLSTM layer 312) by a weight matrix and then adds a bias vector. A softmax layer, such as softmax layer 316 applies a softmax function to the input (e.g., from fully connected layer 314). The softmax function may be used as the last activation function of the neural network classifier (e.g., non-cerebral autoregulation model 126) to normalize the output of fully connected layer 314 to a probability of predicted output classes.

A classification layer such as classification layer 318 uses the probabilities of predicted output classes outputted by softmax layer 316 for the inputs to deep learning architecture 300 to assign the inputs to one of two or more mutually exclusive classes, and may output the output class of the inputs to non-cerebral autoregulation model 126 as a result of training non-cerebral autoregulation model 126 having deep learning architecture 300.

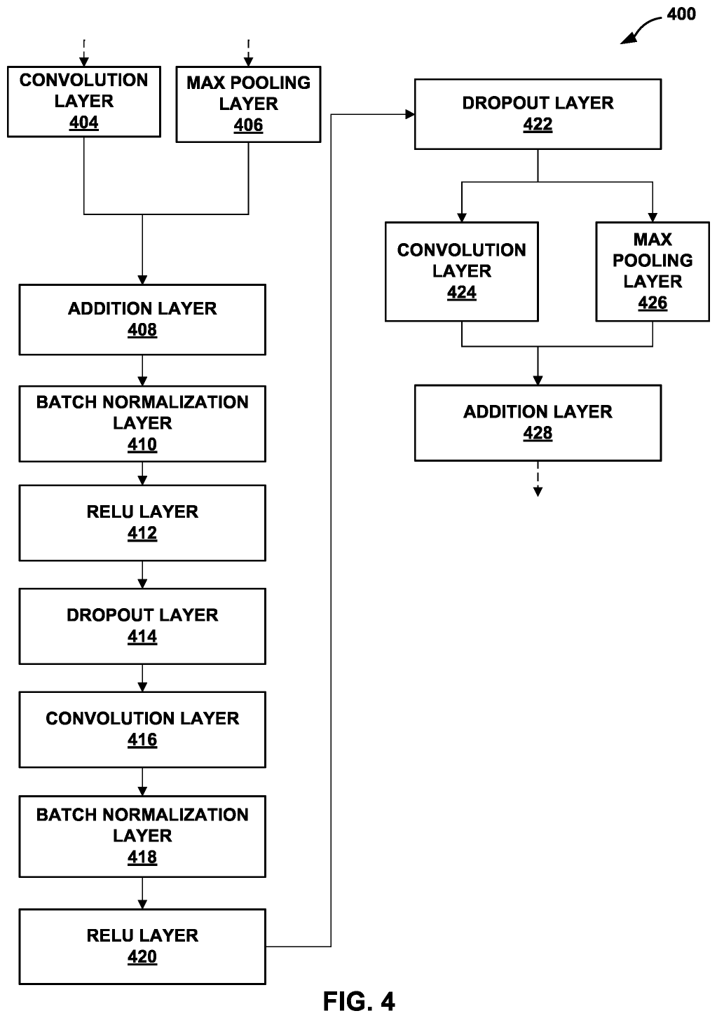

FIG. 4 illustrates an example deep learning architecture 400 of the example non-cerebral autoregulation model 126 of FIGS. 1 and 2. While deep learning architecture 400 is illustrated in FIG. 4 as being a convolutional neural network (CNN) that is used to train a CNN model, any other deep learning architectures may equally be suitable for training non-cerebral autoregulation model 126.

As shown in FIG. 4, a portion of deep learning architecture 400 includes convolution layer 404, max pooling layer 406, addition layer 408, batch normalization layer 410, rectified linear unit (ReLU) layer 412, dropout layer 414, convolution layer 416, batch normalization layer 418, ReLU layer 420, dropout layer 422, convolution layer 424, max pooling layer 426, and addition layer 428. The portion of deep learning architecture 400 illustrated in FIG. 4 may be just a portion (i.e., less than all) of the hidden layers of the CNN that is deep learning architecture 400, and deep learning architecture may include other additional layers not shown in FIG. 4.

A two-dimensional convolution layer, such as convolution layers 404, 416, and 424 applies sliding convolutional filters to the input of the layer. The layer convolves the input by moving the filters along the input vertically and horizontally and computing the dot product of the weights and the input, and then adding a bias term.

A max pooling layer, such as max pooling layers 406 and 426, performs down-sampling by dividing the input into rectangular pooling regions and computing the maximum of each region. A max pooling layer follows convolutional layers for down-sampling, thereby reducing the number of connections to layers that follow the max pooling layer and reduces the number of parameters to be learned in the layers following the max pooling layer. A max pooling layer may also reduce overfitting in the neural network model.

An addition layer, such as addition layers 408 and 428, adds inputs from multiple neural network layers element-wise. In the example of deep learning architecture 400, addition layer 408 may add inputs from convolution layer 404 and max pooling layer 406, and addition layer 428 may add inputs from convolution layer 424 and max pooling layer 426.

A batch normalization layer, such as batch normalization layers 410 and 418, normalizes each input channel across a mini-batch. The batch normalization layer may speed up training of CNNs and reduce sensitivity to network initialization. A batch normalization layer can be used between convolutional layers and ReLU layers, so that batch normalization layer 410 is used in deep learning architecture 400 between convolution layer 404 and ReLU layer 412, and batch normalization layer 418 is used between convolution layer 416 and ReLU layer 420.

A ReLU layer such as ReLU layers 412 and 420, performs a threshold operation to each element of the input, where any value less than zero is set to zero. A ReLU layer may allow faster and more effective training of deep learning architecture 400 on large and complex datasets.

A dropout layer, such as dropout layers 414 and 422, randomly sets input elements to zero with a given probability. By randomly setting input elements to zero, a dropout layer may enable elements to be ignored during the training phase. Selectively ignoring elements during the training phase may prevent over-fitting of training data.

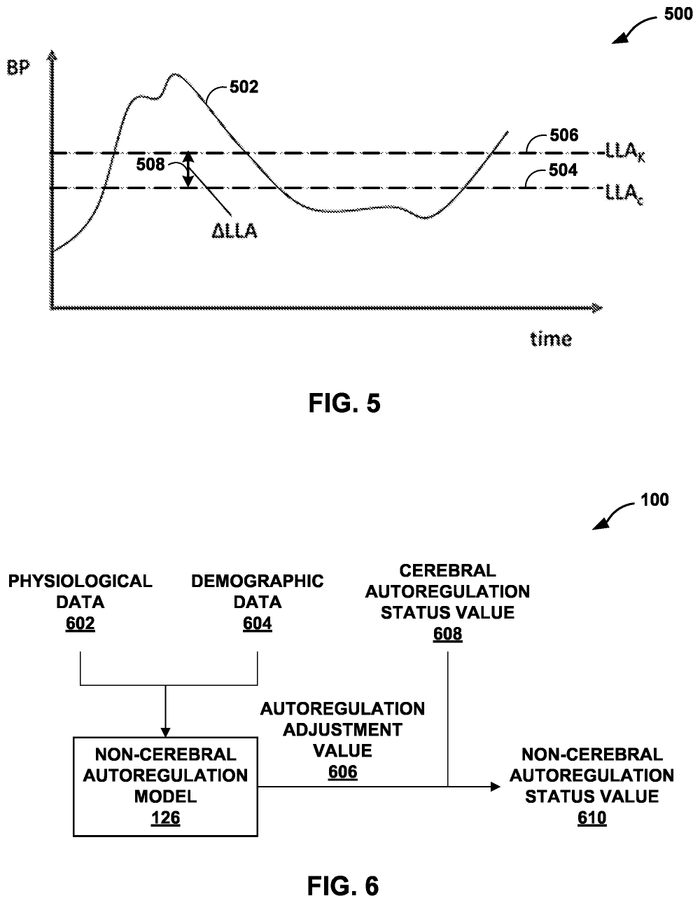

FIG. 5 illustrates an example graph 500 that illustrates a correspondence between the cerebral autoregulation status value of a patient and the kidney autoregulation status value of the patient. In some examples, graph 500 may be an example of a graphical user interface (GUI) that processing circuitry 110 of autoregulation monitoring system 100 may output for display at display 132 to provide the autoregulation status of patient 101 that can be viewed by patient 101 and/or a clinician to monitor the autoregulation status of patient 101.

As shown in FIG. 5, graph 500 illustrates the blood pressure of patient 101 of FIG. 1, over time. Graph 500 also illustrates cerebral LLA 504 of patient 101 and kidney LLA 506 of patient 101. The difference between kidney LLA 506 and cerebral LLA 504 may be LLA adjustment value 508 of patient 101. As can be seen, autoregulation monitoring system 100 may be able to determine kidney LLA 506 of patient 101 based on cerebral LLA 504 of patient 101 by determining LLA adjustment value 508 of patient 101, such as by using non-cerebral autoregulation model 126. Autoregulation monitoring system 100 may therefore determine the kidney LLA 506 of patient 101 as the sum of the LLA adjustment value 508 of patient 101 and cerebral LLA 504 of patient 101.

The LLA of a patient, such as the kidney LLA and/or another non-cerebral LLA of the patient, may, in certain cases, be of greater interest to a clinician than the ULA of the patient because the patient having a high blood pressure may be relatively rare occurrence in a clinical environment and can be managed when it occurs to reduce the blood pressure of the patient. During surgery of a patient, a surgeon or another clinician may control the blood pressure of the patient to keep the blood pressure of the patient at a relatively low level, such as to minimize the chance of bleeding in the patient. However, keeping the blood pressure of the patient at a relatively low level may inadvertently cause the blood pressure of the patient to cross below the LLA of the patient. By determining an LLA for a patient that is individualized for the patient, a clinician may be more accurately determine when the blood pressure of the patient has crossed below the LLA of the patient.

FIG. 6 is a block diagram that illustrates the autoregulation monitoring device 100 of FIG. 1 determining a non-cerebral autoregulation status value for a patient 101. The non-cerebral autoregulation status value for patient 101 may be an autoregulation status value of the kidneys for patient 101, such as the kidney LLA and/or kidney ULA for patient 101.

As shown in FIG. 6, non-cerebral autoregulation model 126 of autoregulation monitoring device 100 may receive, as inputs, physiological data 602 associated with patient 101 and demographic data 604 of patient 101 and may determine, based at least in part on physiological data 602 associated with patient 101 and demographic data 604 of patient 101, autoregulation adjustment value 606 for patient 101. In the example of autoregulation monitoring device 100 determining a kidney LLA for patient 101, autoregulation adjustment value 606 may be a kidney LLA adjustment value for patient 101 with which processing circuitry 110 or other processing circuitry can monitor kidney function of patient 101 based on a cerebral autoregulation status of patient 101.

Physiological data 602 associated with patient 101 may include data such as a blood pressure of patient 101, such as the MAP of patient 101, over a period of time, a regional cerebral oxygen saturation of patient 101 over the period of time, a regional renal oxygen saturation of patient 101 over the period of time, a gradient of the blood pressure of patient 101 over the period of time, a gradient of the regional cerebral oxygen saturation of patient 101 over the period of time, a gradient of the regional renal oxygen saturation of patient 101 over the period of time, a cerebral oxygenation index (COx) of patient 101 over the period of time, a bypass flag indicating that patient 101 was undergoing a cardiopulmonary bypass procedure during the period of time, a first one or more morphology characteristics of a blood pressure signal of patient 101 during the period of time, a second one or more morphology characteristics of a regional cerebral oxygen saturations signal during the period of time, a urine flow rate of patient 101, or urine oxygenation levels of patient 101.

Demographic data 604 patient 101 may include data regarding patient 101's age, sex, height, weight, body mass index, disease state, and the like, as well as, in the case of determining the autoregulation status value of the kidneys for patient 101, data associated with laboratory analysis for serum creatine, calcium, pH, or oxygen level of urine output from patient 101, or other relevant measurements of patient 101's kidney function taken at different periods in time.

Autoregulation monitoring device 100 may determine or otherwise receive cerebral autoregulation status value 608 of patient 101. In the example of autoregulation monitoring device 100 determining a kidney LLA for patient 101, cerebral autoregulation status value 608 for patient 101 may be a cerebral LLA for patient 101.

Autoregulation monitoring device 100 may determine non-cerebral autoregulation status value 610 for patient 101 as a function of autoregulation adjustment value 606 for patient 101 and cerebral autoregulation status value 608 of patient 101. In the example of autoregulation monitoring device 100 determining the autoregulation status value of the kidneys for patient 101, such as the kidney LLA for patient 101, autoregulation monitoring device 100 may determine the kidney LLA for patient 101 as a function of the kidney LLA adjustment value for patient 101 and the cerebral LLA for patient 101, such as the sum of the kidney LLA adjustment value for patient 101 and the cerebral LLA for patient 101.

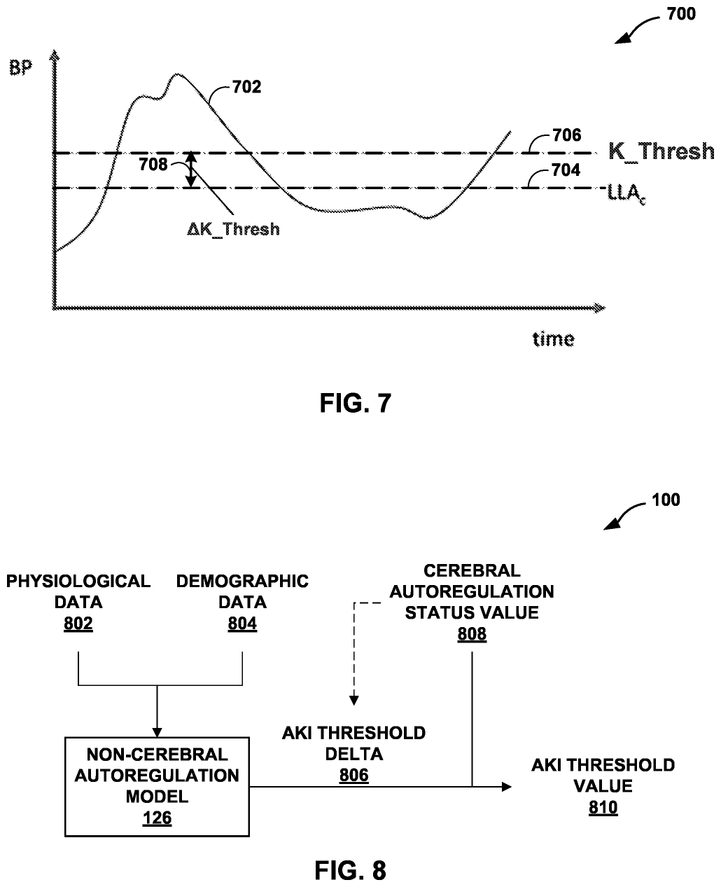

FIG. 7 illustrates an example graph 700 that illustrates an AKI threshold for a patient. In some examples, graph 700 may be an example of a graphical user interface (GUI) that processing circuitry 110 of autoregulation monitoring system 100 may output for display at display 132 to provide the autoregulation status of patient 101 that can be viewed by patient 101 and/or a clinician to monitor whether the blood pressure of patient 101 is associated with an increased risk of developing an acute kidney injury.

As shown in FIG. 7, graph 700 illustrates the blood pressure of patient 101 of FIG. 1, over time. Graph 700 also illustrates cerebral LLA 704 of patient 101 and AKI threshold 706 for patient 101. AKI threshold value 706 may be a blood pressure threshold at which patient 101 may enter an undesirable blood pressure zone, such as a blood pressure associated with an increased risk of developing acute kidney injury, when the blood pressure of patient 101 reaches or crosses (i.e., rises above) AKI threshold value 706.

The difference between AKI threshold value 706 and cerebral LLA 704 may be AKI threshold delta value 708 of patient 101. As can be seen, autoregulation monitoring system 100 may be able to determine AKI threshold value 706 for patient 101 based on cerebral LLA 704 of patient 101 by at least determining AKI threshold delta value 708 of patient 101, such as by using non-cerebral autoregulation model 126. Autoregulation monitoring system 100 may therefore determine AKI threshold value 706 for patient 101 as the sum of the AKI threshold delta value 708 of patient 101 and cerebral LLA 704 of patient 101.

FIG. 8 is a block diagram that illustrates the autoregulation monitoring device 100 of FIG. 1 determining an AKI threshold for a patient 101. The AKI threshold for patient 101. As described above, the AKI threshold for patient 101 may be a blood pressure threshold at which patient 101 may enter an undesirable blood pressure zone, such as a blood pressure associated with an increased risk of developing acute kidney injury, when the blood pressure of patient 101 reaches or crosses (i.e., rises above) AKI threshold

As shown in FIG. 8, non-cerebral autoregulation model 126 of autoregulation monitoring device 100 may receive, as inputs, physiological data 802 associated with patient 101 and demographic data 804 of patient 101 and may determine, based at least in part on physiological data 802 associated with patient 101 and demographic data 804 of patient 101, AKI autoregulation threshold delta value 806 for patient 101.

Autoregulation monitoring device 100 may determine or otherwise receive cerebral autoregulation status value 808 of patient 101, such as the cerebral LLA for patient 101. In some examples, autoregulation monitoring device 100 may determine AKI autoregulation threshold delta value 806 for patient 101 based on both non-cerebral autoregulation model 126 and cerebral autoregulation status value 808 of patient 101, such as the cerebral LLA for patient 101. In some examples, autoregulation monitoring device 100 may use non-cerebral autoregulation model 126 to determine AKI autoregulation threshold delta value 806 for patient 101 and may adjust the determined AKI autoregulation threshold delta value 806 based on cerebral autoregulation status value 808 of patient 101. For example, autoregulation monitoring device 100 may increase or decrease the determined AKI autoregulation threshold delta value 806 based on cerebral autoregulation status value 808 of patient 101, such as by increasing or decreasing the determined AKI autoregulation threshold delta value 806 based on cerebral autoregulation status value 808 of patient 101 being at or above a cerebral autoregulation status value threshold, or by increasing or decreasing the determined AKI autoregulation threshold delta value 806 based on cerebral autoregulation status value 808 of patient 101 being below a cerebral autoregulation status value threshold.

Autoregulation monitoring device 100 may determine AKI autoregulation threshold value 810 for patient 101 as a function of AKI autoregulation threshold delta value 806 for patient 101 and cerebral autoregulation status value 808 of patient 101. For example, autoregulation monitoring device 100 may determine AKI autoregulation threshold value 810 for patient 101 as a function of AKI autoregulation threshold delta value 806 for patient 101 and the cerebral LLA for patient 101, such as the sum of the AKI threshold value 810 for patient 101 and the cerebral LLA for patient 101.

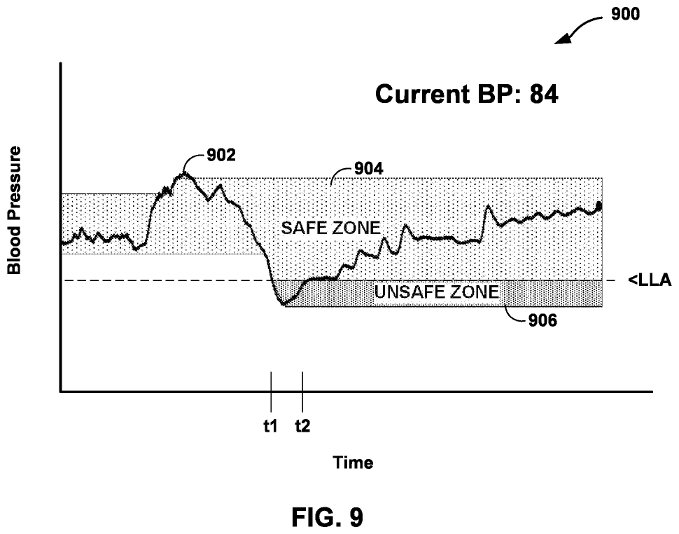

FIG. 9 illustrates example user interface that includes autoregulation information. As shown in FIG. 9, graphical user interface (GUI) 900 is an example of an interface that processing circuitry 110 of autoregulation monitoring system 100 may output for display at display 132 to provide the autoregulation status of patient 101. GUI 900 includes a graph of blood pressure value 902 of patient 101 over time. The LLA shown in FIG. 9 can be, for example, a cerebral LLA, a non-cerebral LLA (e.g., LLA for a kidney or a gastrointestinal tract of patient 101).

Safe zone 904 in GUI 900 may illustrate a zone that is above the LLA, while unsafe zone 909 in GUI 900 may illustrate a zone that is below the LLA. GUI 900 illustrates the relationship between blood pressure value 902 of patient 101 and the autoregulation status of patient 101 by illustrating whether blood pressure value 902 of patient 101 is within safe zone 904 or unsafe zone 909. As illustrated in GUI 900, blood pressure value 902 is in safe zone 904 until blood pressure value 902 drops below the LLA to unsafe zone 909 at time t1, until the blood pressure value 902 returns to being above the LLA and therefore in safe zone 904 at time t2.

In some examples, GUI 900 can present both the cerebral LLA (and/or ULA) and the non-cerebral LLA (and/or ULA) in the same graph, where the cerebral LLA and the non-cerebral LLA are specific to patient 101. In some examples, GUI 900 may present the cerebral LLA and/or the non-cerebral LLA without presenting the cerebral ULA and the non-cerebral ULA. This may enable a clinician to monitor both the brain and the non-cerebral organ (e.g., the kidney) at the same time and using the same display. This simplified display may enable a clinician to more quickly ascertain the autoregulation status of multiple organs of patient 101, which can result in quicker intervention should the blood pressure of patient 101 drop below the displayed cerebral LLA and the non-cerebral LLA. In addition, displaying both the cerebral and non-cerebral LLA can enable a clinician to titrate therapy (e.g., fluid delivery) delivered to patient 101 based on the particular organ of interest and based on the particular needs of patient 101. For example, if the clinician determines that patient 101 is at particular risk of developing AKI or already suffers from relatively low kidney function (e.g., as a result of kidney disease), then the clinician may titrate therapy based on the kidney LLA (and/or the kidney ULA).

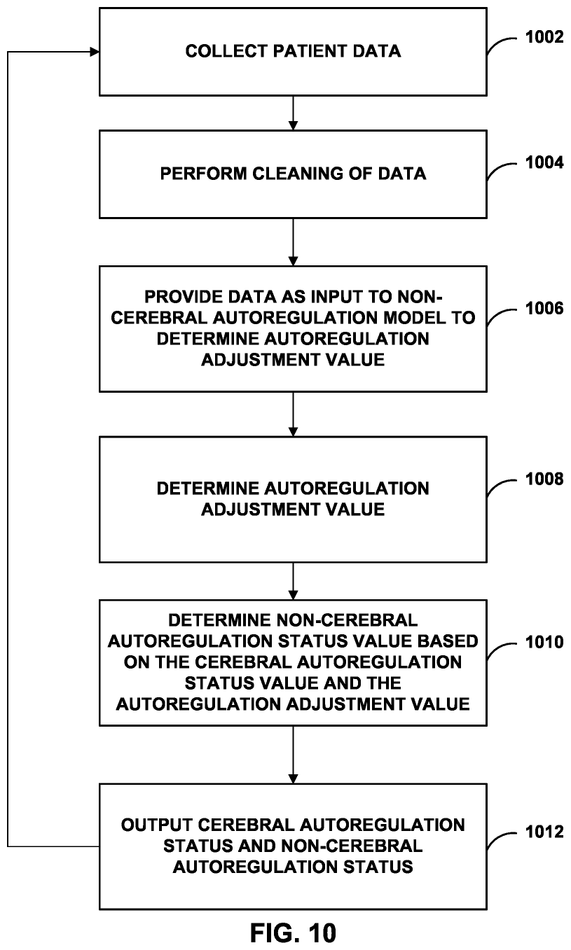

FIG. 10 is a flow diagram illustrating an example method for monitoring the autoregulation status of a patient. Although FIG. 10 is described with respect to processing circuitry 110 of autoregulation monitoring system 100 (FIG. 1), in other examples, different processing circuitry, alone or in combination with processing circuitry 110, may perform any part of the technique of FIG. 10.

As shown in FIG. 10, the method includes processing circuitry 110 collecting patient data regarding patient 101 (1002). To collect the patient data, processing circuitry 110 may receive or otherwise determine physiological data of patient 101. In some examples, collecting patient data may also include collecting demographic data of patient 101. Demographic data of patient 101 may include data regarding patient 101's age, sex, height, weight, body mass index, disease state, and the like, as well as, in the case of determining the autoregulation status value of the kidneys for patient 101, data associated with laboratory analysis for serum creatine, calcium, pH, or other relevant measurements of patient 101's kidney function taken at different periods in time.

In some examples, processing circuitry 110 performs cleaning of the collected patient data (1004). For example, processing circuitry 110 may remove invalid value, such as values for any part of the data that are outside a range of valid values, performing smoothing of the data values, performing interpolation of the values, and the like.

Processing circuitry 110 provides the collected patient data, e.g., the patient data that has been cleaned, to non-cerebral autoregulation model 126 (1006). Processing circuitry 110 executes non-cerebral autoregulation model 126 to output, based on the inputted data, an indication of an autoregulation adjustment value (1008). For example, the autoregulation adjustment value may be in the form of units of millimeters of mercury (mmHg). Such an autoregulation adjustment value may indicate a difference between a cerebral autoregulation status value of patient 101 and an autoregulation status value for a non-cerebral organ of patient 101, such as the kidneys of patient 101.

Processing circuitry 110 may determine an autoregulation status value for a non-cerebral organ of patient 101, such as the autoregulation status value of the kidneys of patient 101, based at least in part on the determined autoregulation adjustment value and a cerebral autoregulation status value for patient 101 (1010). For example, processing circuitry 110 may execute cerebral autoregulation model 124 to determine a cerebral autoregulation status value for patient 101, such as a cerebral LLA or a cerebral ULA for patient 101. Processing circuitry 110 may therefore determine an autoregulation status value of the kidneys of patient 101, such as a kidney LLA, as the sum of the cerebral LLA and the autoregulation adjustment value. In another example, processing circuitry may determine a kidney ULA as the sum of the cerebral ULA and the autoregulation adjustment value.

Processing circuitry 110 may, in response to determining the autoregulation status value of a non-cerebral organ of patient 101, output an indication of the autoregulation status value of a non-cerebral organ of patient 101, such as for display at display 132 (1012). For example, processing circuitry 110 may output a graphical user interface which may provide the currently determined cerebral autoregulation status value of patient 101 along with the autoregulation status value of a non-cerebral organ of patient 101, as well as additional information associated with patient 101, such as the blood pressure of patient 101, the regional cerebral oxygen saturation of patient 101, and the like.

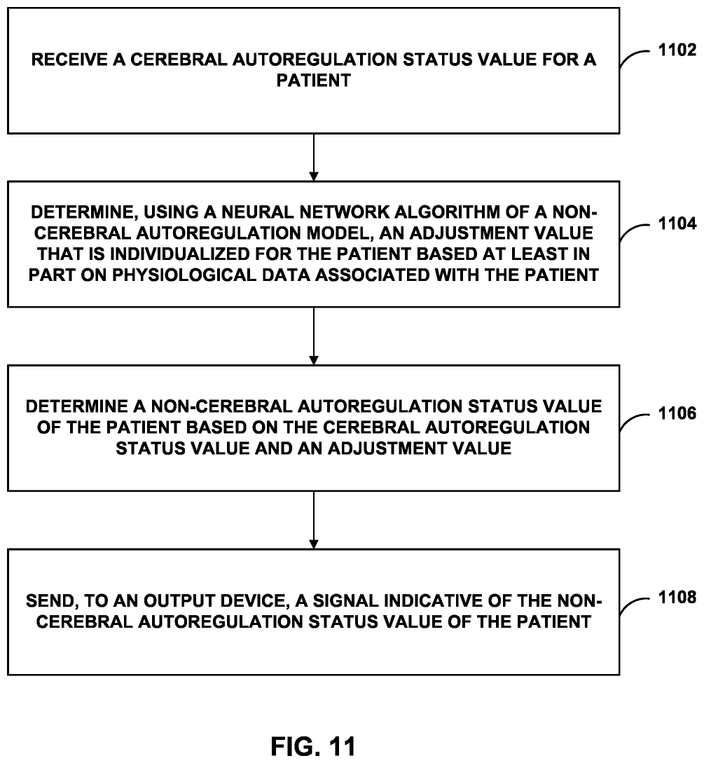

FIG. 11 is a flow diagram illustrating an example method for monitoring the autoregulation status of a patient. Although FIG. 11 is described with respect to processing circuitry 110 of autoregulation monitoring system 100 (FIG. 1), in other examples, different processing circuitry, alone or in combination with processing circuitry 110, may perform any part of the technique of FIG. 11.

As shown in FIG. 11, processing circuitry 110 of cerebral autoregulation monitoring system 100 may receive a cerebral autoregulation status value for a patient 101 (1102). Processing circuitry 110 may determine, using a neural network algorithm of a non-cerebral autoregulation model 126, an adjustment value that is individualized for the patient based at least in part on physiological data associated with the patient (1104). Processing circuitry 110 may determine a non-cerebral autoregulation status value of the patient 101 based on the cerebral autoregulation status value and the adjustment value (1106). Processing circuitry 110 may send, to an output device, a signal indicative of the non-cerebral autoregulation status value of the patient 101 (1108).

This disclosure includes the following examples:

Example 1: A method includes receiving, by processing circuitry, a cerebral autoregulation status value for a patient; determining, by the processing circuitry and using a neural network algorithm of a non-cerebral autoregulation model, an adjustment value that is individualized for the patient based at least in part on physiological data associated with the patient; determining, by the processing circuitry, a non-cerebral autoregulation status value of the patient based on the cerebral autoregulation status value and the adjustment value; and sending, by the processing circuitry and to an output device, a signal indicative of the non-cerebral autoregulation status value of the patient.

Example 2: The method of example 1, further includes determining, by the processing circuitry and using the neural network algorithm of the non-cerebral autoregulation model, an acute kidney threshold delta value that is individualized for the patient; and determining, by the processing circuitry, an acute kidney injury threshold value for the patient based on the cerebral autoregulation status value, the adjustment value, and the acute kidney threshold delta value.

Example 3: The method of any of examples 1 and 2, wherein determining, using the neural network algorithm of the non-cerebral autoregulation model, the adjustment value further comprises inputting, by the processing circuitry, the physiological data associated with the patient and demographic data associated with the patient into the non-cerebral autoregulation model.

Example 4: The method of any of examples 1-3, wherein the physiological data associated with the patient comprises one or more of: a blood pressure of the patient over a period of time, a regional cerebral oxygen saturation of the patient over the period of time, a regional renal oxygen saturation of the patient over the period of time, a gradient of the blood pressure of the patient over the period of time, a gradient of the regional cerebral oxygen saturation of the patient over the period of time, a gradient of the regional renal oxygen saturation of the patient over the period of time, a cerebral oxygenation index of the patient over the period of time, a bypass flag indicating that the patient was undergoing a cardiopulmonary bypass procedure during the period of time, a first one or more morphology characteristics of a blood pressure signal of the patient during the period of time, a second one or more morphology characteristics of a regional cerebral oxygen saturation signal during the period of time, a urine flow rate of the patient, or a urine oxygenation level of the patient.

Example 5: The method of example 3, wherein the demographic data associated with the patient comprises one or more of: a height of the patient, a weight of the patient, an age of the patient, a body mass index of the patient, a disease state of the patient, or laboratory analysis results for one or more measurements of kidney function of the patient.

Example 6: The method of any of examples 1-5, wherein the neural network algorithm is trained via machine learning over training data that includes one or more of: blood pressures of one or more patients over time; regional cerebral oxygen saturation values of the one or more patients over the time; regional renal oxygen saturation values of the one or more patients over the time; gradients of the blood pressures of the one or more patients over each of a plurality of time periods; gradients of the regional cerebral oxygen saturation values of the one or more patients over each of the plurality of time periods; gradients of the regional renal oxygen saturation values of the one or more patients over each of the plurality of time periods; cerebral oxygenation indices (COx) determined based on the blood pressures and the regional cerebral oxygen saturations of the one or more patients over each of the time periods; one or more bypass flags indicating whether the one or more patients were undergoing a cardiopulmonary bypass procedure during each of the time periods;
morphology characteristics of at least one sensor signal indicative of at least one of the one or more of the blood pressures, the regional cerebral oxygen saturation values, or the regional renal oxygen saturation values during each of the time periods; urine flow rates of the one or more patients; urine oxygenation levels of the one or more patient; systolic blood pressures of the one or more patients over time; diastolic blood pressures of the one or more patients over time; or demographic data associated with the one or more patients.

Example 7: The method of example 6, wherein: the blood pressures of the one or more patients over time comprise, for each of the time periods, the blood pressures during a respective time period minus a mean of the blood pressures over time, the regional cerebral oxygen saturation values of the one or more patients over time comprise, for each of the time periods, the regional cerebral oxygen saturation values during the respective time period minus a mean of the regional cerebral oxygen saturation values over time, and the regional renal oxygen saturation values of the one or more patients over time comprise, for each of the time periods, the regional renal oxygen saturation values during the respective time period minus a mean of the regional renal oxygen saturation values over time.

Example 8: The method of any of examples 1-7, further comprising presenting, via a display of the output device, a user interface indicating the cerebral autoregulation status value and the non-cerebral autoregulation status value.

Example 9: The method of any of examples 1-8, wherein the non-cerebral autoregulation status value of the patient comprises an autoregulation status value of the kidneys of the patient.

Example 10: A system includes memory; and processing circuitry operably coupled to the memory and configured to: receive a cerebral autoregulation status value for a patient; determine, using a neural network algorithm of a non-cerebral autoregulation model, an adjustment value that is individualized for the patient based at least in part on physiological data associated with the patient; determine a non-cerebral autoregulation status value of the patient based on the cerebral autoregulation status value and the adjustment value; and send, to an output device, a signal indicative of the non-cerebral autoregulation status value of the patient.

Example 11: The system of example 10, wherein the processing circuitry is further configured to: determine, using the neural network algorithm of the non-cerebral autoregulation model, an acute kidney threshold delta value that is individualized for the patient; and determine an acute kidney injury threshold value for the patient based on the cerebral autoregulation status value, the adjustment value, and the acute kidney threshold delta value.

Example 12: The system of any of examples 10 and 11, wherein to determine, using the neural network algorithm of the non-cerebral autoregulation model, the adjustment value, the processing circuitry is further configured to input the physiological data associated with the patient and demographic data associated with the patient into the non-cerebral autoregulation model.

Example 13: The system of any of examples 10-12, wherein the physiological data associated with the patient comprises one or more of: a blood pressure of the patient over a period of time, a regional cerebral oxygen saturation of the patient over the period of time, a regional renal oxygen saturation of the patient over the period of time, a gradient of the blood pressure of the patient over the period of time, a gradient of the regional cerebral oxygen saturation of the patient over the period of time, a gradient of the regional renal oxygen saturation of the patient over the period of time, a cerebral oxygenation index of the patient over the period of time, a bypass flag indicating that the patient was undergoing a cardiopulmonary bypass procedure during the period of time, a first one or more morphology characteristics of a blood pressure signal of the patient during the period of time, a second one or more morphology characteristics of a regional cerebral oxygen saturation signal during the period of time, a urine flow rate of the patient, or a urine oxygenation level of the patient.

Example 14: The system of example 12, wherein the demographic data associated with the patient comprises one or more of: a height of the patient, a weight of the patient, an age of the patient, a body mass index of the patient, a disease state of the patient, or laboratory analysis results for one or more measurements of kidney function of the patient.

Example 15: The system of any of examples 10-14, wherein the neural network algorithm is trained via machine learning over training data that includes one or more of: blood pressures of one or more patients over time; regional cerebral oxygen saturation values of the one or more patients over the time; regional renal oxygen saturation values of the one or more patients over the time; gradients of the blood pressures of the one or more patients over each of a plurality of time periods; gradients of the regional cerebral oxygen saturation values of the one or more patients over each of the plurality of time periods; gradients of the regional renal oxygen saturation values of the one or more patients over each of the plurality of time periods; cerebral oxygenation indices (COx) determined based on the blood pressures and the regional cerebral oxygen saturations of the one or more patients over each of the time periods; one or more bypass flags indicating whether the one or more patients were undergoing a cardiopulmonary bypass procedure during each of the time periods;
morphology characteristics of at least one sensor signal indicative of at least one of the one or more of the blood pressures, the regional cerebral oxygen saturation values, or the regional renal oxygen saturation values during each of the time periods; urine flow rates of the one or more patients; urine oxygenation levels of the one or more patient; systolic blood pressures of the one or more patients over time; diastolic blood pressures of the one or more patients over time; or demographic data associated with the one or more patients.

Example 16: The system of any of examples 10-15, wherein: the blood pressures of the one or more patients over time comprise, for each of the time periods, the blood pressures during a respective time period minus a mean of the blood pressures over time, the regional cerebral oxygen saturation values of the one or more patients over time comprise, for each of the time periods, the regional cerebral oxygen saturation values during the respective time period minus a mean of the regional cerebral oxygen saturation values over time, and the regional renal oxygen saturation values of the one or more patients over time comprise, for each of the time periods, the regional renal oxygen saturation values during the respective time period minus a mean of the regional renal oxygen saturation values over time.

Example 17: The system of any of examples 10-16, wherein the processing circuitry is further configured to present, via a display of the output device, a user interface indicating the cerebral autoregulation status value and the non-cerebral autoregulation status value.

Example 18: The system of any of examples 10-17, wherein the non-cerebral autoregulation status value of the patient comprises an autoregulation status value of the kidneys of the patient.

Example 19: A non-transitory computer readable storable medium includes receive a cerebral autoregulation status value for a patient; determine, using a neural network algorithm of a non-cerebral autoregulation model, an adjustment value that is individualized for the patient based at least in part on physiological data associated with the patient; determine a non-cerebral autoregulation status value of the patient based on the cerebral autoregulation status value and the adjustment value; and send, to an output device, a signal indicative of the non-cerebral autoregulation status value of the patient.

Example 20: The non-transitory computer readable storable medium of example 19, wherein the instructions further cause the processing circuitry to: determine, using the neural network algorithm of the non-cerebral autoregulation model, an acute kidney threshold delta value; and determine an acute kidney injury threshold value for the patient based on the cerebral autoregulation status value, the adjustment value, and the acute kidney threshold delta value.

The techniques described in this disclosure, including those attributed to system 100, processing circuitry 110, control circuitry 122, sensing circuitries 140, 142, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as clinician or patient programmers, medical devices, or other devices. Processing circuitry, control circuitry, and sensing circuitry, as well as other processors and controllers described herein, may be implemented at least in part as, or include, one or more executable applications, application modules, libraries, classes, methods, objects, routines, subroutines, firmware, and/or embedded code, for example.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. The computer-readable medium may be an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disk ROM (CD-ROM), a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

The functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements.

The following clauses include example subject matter described herein.

Clause 1: A method includes receiving, by processing circuitry, a cerebral autoregulation status value for a patient; determining, by the processing circuitry and using a neural network algorithm of a non-cerebral autoregulation model, an adjustment value that is individualized for the patient based at least in part on physiological data associated with the patient; determining, by the processing circuitry, a non-cerebral autoregulation status value of the patient based on the cerebral autoregulation status value and the adjustment value; and sending, by the processing circuitry and to an output device, a signal indicative of the non-cerebral autoregulation status value of the patient.

Clause 2: The method of clause 1, further includes determining, by the processing circuitry and using the neural network algorithm of the non-cerebral autoregulation model, an acute kidney threshold delta value that is individualized for the patient; and determining, by the processing circuitry, an acute kidney injury threshold value for the patient based on the cerebral autoregulation status value, the adjustment value, and the acute kidney threshold delta value.

Clause 3: The method of any of clauses 1 and 2, wherein determining, using the neural network algorithm of the non-cerebral autoregulation model, the adjustment value further comprises inputting, by the processing circuitry, the physiological data associated with the patient and demographic data associated with the patient into the non-cerebral autoregulation model.

Clause 4: The method of any of clauses 1-3, wherein the physiological data associated with the patient comprises one or more of: a blood pressure of the patient over a period of time, a regional cerebral oxygen saturation of the patient over the period of time, a regional renal oxygen saturation of the patient over the period of time, a gradient of the blood pressure of the patient over the period of time, a gradient of the regional cerebral oxygen saturation of the patient over the period of time, a gradient of the regional renal oxygen saturation of the patient over the period of time, a cerebral oxygenation index of the patient over the period of time, a bypass flag indicating that the patient was undergoing a cardiopulmonary bypass procedure during the period of time, a first one or more morphology characteristics of a blood pressure signal of the patient during the period of time, a second one or more morphology characteristics of a regional cerebral oxygen saturation signal during the period of time, a urine flow rate of the patient, or a urine oxygenation level of the patient.

Clause 5: The method of any of clauses 3 and 4, wherein the demographic data associated with the patient comprises one or more of: a height of the patient, a weight of the patient, an age of the patient, a body mass index of the patient, a disease state of the patient, or laboratory analysis results for one or more measurements of kidney function of the patient.

Clause 6: The method of any of clauses 1-5, wherein the neural network algorithm is trained via machine learning over training data that includes one or more of: blood pressures of one or more patients over time; regional cerebral oxygen saturation values of the one or more patients over the time; regional renal oxygen saturation values of the one or more patients over the time; gradients of the blood pressures of the one or more patients over each of a plurality of time periods; gradients of the regional cerebral oxygen saturation values of the one or more patients over each of the plurality of time periods; gradients of the regional renal oxygen saturation values of the one or more patients over each of the plurality of time periods; cerebral oxygenation indices (COx) determined based on the blood pressures and the regional cerebral oxygen saturations of the one or more patients over each of the time periods; one or more bypass flags indicating whether the one or more patients were undergoing a cardiopulmonary bypass procedure during each of the time periods; morphology characteristics of at least one sensor signal indicative of at least one of the one or more of the blood pressures, the regional cerebral oxygen saturation values, or the regional renal oxygen saturation values during each of the time periods; urine flow rates of the one or more patients; urine oxygenation levels of the one or more patient; systolic blood pressures of the one or more patients over time; diastolic blood pressures of the one or more patients over time; or demographic data associated with the one or more patients.

Clause 7: The method of clause 6, wherein: the blood pressures of the one or more patients over time comprise, for each of the time periods, the blood pressures during a respective time period minus a mean of the blood pressures over time, the regional cerebral oxygen saturation values of the one or more patients over time comprise, for each of the time periods, the regional cerebral oxygen saturation values during the respective time period minus a mean of the regional cerebral oxygen saturation values over time, and the regional renal oxygen saturation values of the one or more patients over time comprise, for each of the time periods, the regional renal oxygen saturation values during the respective time period minus a mean of the regional renal oxygen saturation values over time.

Clause 8: The method of any of clauses 1-7, further comprising presenting, via a display of the output device, a user interface indicating the cerebral autoregulation status value and the non-cerebral autoregulation status value.

Clause 9: The method of any of clauses 1-8, wherein the non-cerebral autoregulation status value of the patient comprises an autoregulation status value of the kidneys of the patient.

Clause 10: A system includes memory; and processing circuitry operably coupled to the memory and configured to: receive a cerebral autoregulation status value for a patient; determine, using a neural network algorithm of a non-cerebral autoregulation model, an adjustment value that is individualized for the patient based at least in part on physiological data associated with the patient; determine a non-cerebral autoregulation status value of the patient based on the cerebral autoregulation status value and the adjustment value; and send, to an output device, a signal indicative of the non-cerebral autoregulation status value of the patient.

Clause 11: The system of clause 10, wherein the processing circuitry is further configured to: determine, using the neural network algorithm of the non-cerebral autoregulation model, an acute kidney threshold delta value that is individualized for the patient; and determine an acute kidney injury threshold value for the patient based on the cerebral autoregulation status value, the adjustment value, and the acute kidney threshold delta value.

Clause 12: The system of any of clauses 10 and 11, wherein to determine, using the neural network algorithm of the non-cerebral autoregulation model, the adjustment value, the processing circuitry is further configured to input the physiological data associated with the patient and demographic data associated with the patient into the non-cerebral autoregulation model.

Clause 13: The system of any of clauses 10-12, wherein the physiological data associated with the patient comprises one or more of: a blood pressure of the patient over a period of time, a regional cerebral oxygen saturation of the patient over the period of time, a regional renal oxygen saturation of the patient over the period of time, a gradient of the blood pressure of the patient over the period of time, a gradient of the regional cerebral oxygen saturation of the patient over the period of time, a gradient of the regional renal oxygen saturation of the patient over the period of time, a cerebral oxygenation index of the patient over the period of time, a bypass flag indicating that the patient was undergoing a cardiopulmonary bypass procedure during the period of time, a first one or more morphology characteristics of a blood pressure signal of the patient during the period of time, a second one or more morphology characteristics of a regional cerebral oxygen saturation signal during the period of time, a urine flow rate of the patient, or a urine oxygenation level of the patient.

Clause 14: The system of any of clauses 12 and 13, wherein the demographic data associated with the patient comprises one or more of: a height of the patient, a weight of the patient, an age of the patient, a body mass index of the patient, a disease state of the patient, or laboratory analysis results for one or more measurements of kidney function of the patient.

Clause 15: The system of any of clauses 10-14, wherein the neural network algorithm is trained via machine learning over training data that includes one or more of: blood pressures of one or more patients over time; regional cerebral oxygen saturation values of the one or more patients over the time; regional renal oxygen saturation values of the one or more patients over the time; gradients of the blood pressures of the one or more patients over each of a plurality of time periods; gradients of the regional cerebral oxygen saturation values of the one or more patients over each of the plurality of time periods; gradients of the regional renal oxygen saturation values of the one or more patients over each of the plurality of time periods; cerebral oxygenation indices (COx) determined based on the blood pressures and the regional cerebral oxygen saturations of the one or more patients over each of the time periods; one or more bypass flags indicating whether the one or more patients were undergoing a cardiopulmonary bypass procedure during each of the time periods; morphology characteristics of at least one sensor signal indicative of at least one of the one or more of the blood pressures, the regional cerebral oxygen saturation values, or the regional renal oxygen saturation values during each of the time periods; urine flow rates of the one or more patients; urine oxygenation levels of the one or more patient; systolic blood pressures of the one or more patients over time; diastolic blood pressures of the one or more patients over time; or demographic data associated with the one or more patients.

Clause 16: The system of clause 15, wherein: the blood pressures of the one or more patients over time comprise, for each of the time periods, the blood pressures during a respective time period minus a mean of the blood pressures over time, the regional cerebral oxygen saturation values of the one or more patients over time comprise, for each of the time periods, the regional cerebral oxygen saturation values during the respective time period minus a mean of the regional cerebral oxygen saturation values over time, and the regional renal oxygen saturation values of the one or more patients over time comprise, for each of the time periods, the regional renal oxygen saturation values during the respective time period minus a mean of the regional renal oxygen saturation values over time.

Clause 17: The system of any of clauses 10-16, wherein the processing circuitry is further configured to present, via a display of the output device, a user interface indicating the cerebral autoregulation status value and the non-cerebral autoregulation status value.

Clause 18: The system of any of clauses 10-17, wherein the non-cerebral autoregulation status value of the patient comprises an autoregulation status value of the kidneys of the patient.

Clause 19: A non-transitory computer readable storable medium includes receive a cerebral autoregulation status value for a patient; determine, using a neural network algorithm of a non-cerebral autoregulation model, an adjustment value that is individualized for the patient based at least in part on physiological data associated with the patient; determine a non-cerebral autoregulation status value of the patient based on the cerebral autoregulation status value and the adjustment value; and send, to an output device, a signal indicative of the non-cerebral autoregulation status value of the patient.

Clause 20: The non-transitory computer readable storable medium of clause 19, wherein the instructions further cause the processing circuitry to: determine, using the neural network algorithm of the non-cerebral autoregulation model, an acute kidney threshold delta value; and determine an acute kidney injury threshold value for the patient based on the cerebral autoregulation status value, the adjustment value, and the acute kidney threshold delta value.

Various examples of the disclosure have been described. Any combination of the described systems, operations, or functions is contemplated. These and other examples are within the scope of the following claims.

What is claimed is:

1. A method comprising:
receiving, by processing circuitry, a cerebral autoregulation status value for a patient;
determining, by the processing circuitry and using a neural network algorithm of a non-cerebral autoregulation model, an adjustment value that is individualized for the patient based at least in part on physiological data associated with the patient;
determining, by the processing circuitry, a non-cerebral autoregulation status value of the patient based on the cerebral autoregulation status value and the adjustment value; and
sending, by the processing circuitry and to an output device, a signal indicative of the non-cerebral autoregulation status value of the patient.

2. The method of claim 1, further comprising:
determining, by the processing circuitry and using the neural network algorithm of the non-cerebral autoregulation model, an acute kidney threshold delta value that is individualized for the patient; and
determining, by the processing circuitry, an acute kidney injury threshold value for the patient based on the cerebral autoregulation status value, the adjustment value, and the acute kidney threshold delta value.

3. The method of claim 1, wherein determining, using the neural network algorithm of the non-cerebral autoregulation model, the adjustment value further comprises inputting, by the processing circuitry, the physiological data associated with the patient and demographic data associated with the patient into the non-cerebral autoregulation model.

4. The method of claim 1, wherein the physiological data associated with the patient comprises one or more of:
a blood pressure of the patient over a period of time,
a regional cerebral oxygen saturation of the patient over the period of time,
a regional renal oxygen saturation of the patient over the period of time,
a gradient of the blood pressure of the patient over the period of time,
a gradient of the regional cerebral oxygen saturation of the patient over the period of time,
a gradient of the regional renal oxygen saturation of the patient over the period of time,
a cerebral oxygenation index of the patient over the period of time,
a bypass flag indicating that the patient was undergoing a cardiopulmonary bypass procedure during the period of time,
a first one or more morphology characteristics of a blood pressure signal of the patient during the period of time,
a second one or more morphology characteristics of a regional cerebral oxygen saturation signal during the period of time,
a urine flow rate of the patient, or
a urine oxygenation level of the patient.

5. The method of claim 3, wherein the demographic data associated with the patient comprises one or more of:
a height of the patient,
a weight of the patient,
an age of the patient,
a body mass index of the patient,
a disease state of the patient, or
laboratory analysis results for one or more measurements of kidney function of the patient.

6. The method of claim 1, wherein the neural network algorithm is trained via machine learning over training data that includes one or more of:
blood pressures of one or more patients over time;
regional cerebral oxygen saturation values of the one or more patients over the time;
regional renal oxygen saturation values of the one or more patients over the time;
gradients of the blood pressures of the one or more patients over each of a plurality of time periods;
gradients of the regional cerebral oxygen saturation values of the one or more patients over each of the plurality of time periods;
gradients of the regional renal oxygen saturation values of the one or more patients over each of the plurality of time periods;
cerebral oxygenation indices (COx) determined based on the blood pressures and the regional cerebral oxygen saturations of the one or more patients over each of the time periods;
one or more bypass flags indicating whether the one or more patients were undergoing a cardiopulmonary bypass procedure during each of the time periods;
morphology characteristics of at least one sensor signal indicative of at least one of the one or more of the blood pressures, the regional cerebral oxygen saturation values, or the regional renal oxygen saturation values during each of the time periods;
urine flow rates of the one or more patients;
urine oxygenation levels of the one or more patient;
systolic blood pressures of the one or more patients over time;
diastolic blood pressures of the one or more patients over time; or
demographic data associated with the one or more patients.

7. The method of claim 6, wherein:
the blood pressures of the one or more patients over time comprise, for each of the time periods, the blood pressures during a respective time period minus a mean of the blood pressures over time,
the regional cerebral oxygen saturation values of the one or more patients over time comprise, for each of the time periods, the regional cerebral oxygen saturation values during the respective time period minus a mean of the regional cerebral oxygen saturation values over time, and
the regional renal oxygen saturation values of the one or more patients over time comprise, for each of the time periods, the regional renal oxygen saturation values during the respective time period minus a mean of the regional renal oxygen saturation values over time.

8. The method of claim 1, further comprising presenting, via a display of the output device, a user interface indicating the cerebral autoregulation status value and the non-cerebral autoregulation status value.

9. The method of claim 1, wherein the non-cerebral autoregulation status value of the patient comprises an autoregulation status value of the kidneys of the patient.

10. A system comprising:
memory; and
processing circuitry operably coupled to the memory and configured to:
receive a cerebral autoregulation status value for a patient;
determine, using a neural network algorithm of a non-cerebral autoregulation model, an adjustment value that is individualized for the patient based at least in part on physiological data associated with the patient;
determine a non-cerebral autoregulation status value of the patient based on the cerebral autoregulation status value and the adjustment value; and
send, to an output device, a signal indicative of the non-cerebral autoregulation status value of the patient.

11. The system of claim 10, wherein the processing circuitry is further configured to:
determine, using the neural network algorithm of the non-cerebral autoregulation model, an acute kidney threshold delta value that is individualized for the patient; and
determine an acute kidney injury threshold value for the patient based on the cerebral autoregulation status value, the adjustment value, and the acute kidney threshold delta value.

12. The system of claim 10, wherein to determine, using the neural network algorithm of the non-cerebral autoregulation model, the adjustment value, the processing circuitry is further configured to input the physiological data associated with the patient and demographic data associated with the patient into the non-cerebral autoregulation model.

13. The system of claim 10, wherein the physiological data associated with the patient comprises one or more of:
a blood pressure of the patient over a period of time,
a regional cerebral oxygen saturation of the patient over the period of time,
a regional renal oxygen saturation of the patient over the period of time,
a gradient of the blood pressure of the patient over the period of time,
a gradient of the regional cerebral oxygen saturation of the patient over the period of time,
a gradient of the regional renal oxygen saturation of the patient over the period of time,
a cerebral oxygenation index of the patient over the period of time,
a bypass flag indicating that the patient was undergoing a cardiopulmonary bypass procedure during the period of time,
a first one or more morphology characteristics of a blood pressure signal of the patient during the period of time,
a second one or more morphology characteristics of a regional cerebral oxygen saturation signal during the period of time,
a urine flow rate of the patient, or
a urine oxygenation level of the patient.

14. The system of claim 12, wherein the demographic data associated with the patient comprises one or more of:
a height of the patient,
a weight of the patient,
an age of the patient,
a body mass index of the patient,
a disease state of the patient, or
laboratory analysis results for one or more measurements of kidney function of the patient.

15. The system of claim 10, wherein the neural network algorithm is trained via machine learning over training data that includes one or more of:
blood pressures of one or more patients over time;
regional cerebral oxygen saturation values of the one or more patients over the time;
regional renal oxygen saturation values of the one or more patients over the time;
gradients of the blood pressures of the one or more patients over each of a plurality of time periods;
gradients of the regional cerebral oxygen saturation values of the one or more patients over each of the plurality of time periods;
gradients of the regional renal oxygen saturation values of the one or more patients over each of the plurality of time periods;
cerebral oxygenation indices (COx) determined based on the blood pressures and the regional cerebral oxygen saturations of the one or more patients over each of the time periods;
one or more bypass flags indicating whether the one or more patients were undergoing a cardiopulmonary bypass procedure during each of the time periods;
morphology characteristics of at least one sensor signal indicative of at least one of the one or more of the blood pressures, the regional cerebral oxygen saturation values, or the regional renal oxygen saturation values during each of the time periods;
urine flow rates of the one or more patients;
urine oxygenation levels of the one or more patient;
systolic blood pressures of the one or more patients over time;
diastolic blood pressures of the one or more patients over time; or
demographic data associated with the one or more patients.

16. The system of claim 15, wherein:
the blood pressures of the one or more patients over time comprise, for each of the time periods, the blood pressures during a respective time period minus a mean of the blood pressures over time,
the regional cerebral oxygen saturation values of the one or more patients over time comprise, for each of the time periods, the regional cerebral oxygen saturation values during the respective time period minus a mean of the regional cerebral oxygen saturation values over time, and
the regional renal oxygen saturation values of the one or more patients over time comprise, for each of the time periods, the regional renal oxygen saturation values during the respective time period minus a mean of the regional renal oxygen saturation values over time.

17. The system of claim 10, wherein the processing circuitry is further configured to present, via a display of the output device, a user interface indicating the cerebral autoregulation status value and the non-cerebral autoregulation status value.

18. The system of claim 10, wherein the non-cerebral autoregulation status value of the patient comprises an autoregulation status value of the kidneys of the patient.

19. A non-transitory computer readable storable medium comprising instructions that, when executed, cause processing circuitry to:
receive a cerebral autoregulation status value for a patient;
determine, using a neural network algorithm of a non-cerebral autoregulation model, an adjustment value that is individualized for the patient based at least in part on physiological data associated with the patient;
determine a non-cerebral autoregulation status value of the patient based on the cerebral autoregulation status value and the adjustment value; and
send, to an output device, a signal indicative of the non-cerebral autoregulation status value of the patient.

20. The non-transitory computer readable storable medium of claim 19, wherein the instructions further cause the processing circuitry to:
determine, using the neural network algorithm of the non-cerebral autoregulation model, an acute kidney threshold delta value; and
determine an acute kidney injury threshold value for the patient based on the cerebral autoregulation status value, the adjustment value, and the acute kidney threshold delta value.
